SEQUENCE LISTING (40 sequences)

SEQ ID NO: 1 - protein, 413 aa, Buttiauxella sp.
NDTPASGYQVEKVVILSRHGVRAPTKMTQTMRDVTPNTWPEWPVKLGYITPRGEHLISLMGGFYRQKFQQQGILSQGSCPTPNSIYVWTDVAQRTLKTGEAFLAGLAPQCGLTIHHQQNLEKADPLFHPVKAGICSMDKTQVQQAVEKEAQTPIDNLNQHYIPSLALMNTTLNFSKSPWCQKHSADKSCDLGLSMPSKLSIKDNGNEVSLDGAIGLSSTLAEIFLLEYAQGMPQAAWGNIHSEQEWALLLKLHNVYFDLMERTPYIARHKGTPLLQAISNALNPNATESKLPDISPDNKILFIAGHDTNIANIAGMLNMRWTLPGQPDNTPPGGALVFERLADKSGKQYVSVSMVYQTLEQLRSQTPLSLNQPAGSVQLKIPGCNDQTAEGYCPLSTFTRVVSQSVEPGCQLQ

SEQ ID NO: 2 - protein, 316 aa, Trichoderma resei
MRSSPVLRTALAAALPLAALAADGKSTRYWDCCKPSCSWPGKASVNQPVFACSANFQRISDPNVKSGCDGGSAYACADQTPWAVNDNFSYGFAATSISGGNEASWCCGCYELTFTSGPVAGKTMVVQSTSTGGDLGTNHFDLAMPGGGVGIFDGCSPQFGGLAGDRYGGVSSRSQCDSFPAALKPGCYWRFDWFKNADNPTFTFRQVQCPSELVARTGCRRNDDGNFPVFTPPSGGQSSSSSSSSSAKPTSTSTSTTSTKATSTTSTASSQTSSSTGGGCAAQRWAQCGGIGFSGCTTCVSGTTCNKQNDWYSQCL

SEQ ID NO: 3 - protein, 433 aa, Artificial Sequence
  Synthetic
MSTFIIRLLFFSLLCGSFSIHAEEQNGMKLERVVIVSRHGVRAPTKFTPIMKNVTPDQWPQWDVPLGWLTPRGGELVSELGQYQRLWFTSKGLLNNQTCPSPGQVAVIADTDQRTRKTGEAFLAGLAPKCQIQVHYQKDEEKNDPLFNPVKMGKCSFNTLQVKNAILERAGGNIELYTQRYQSSFRTLENVLNFSQSETCKTTEKSTKCTLPEALPSELKVTPDNVSLPGAWSLSSTLTEIFLLQEAQGMPQVAWGRITGEKEWRDLLSLHNAQFDLLQRTPEVARSRATPLLDMIDTALLTNGTTENRYGIKLPVSLLFIAGHDTNLANLSGALDLNWSLPGQPDNTPPGGELVFEKWKRTSDNTDWVQVSFVYQTLRDMRDIQPLSLEKPAGKVDLKLIACEEKNSQGMCSLKSFSRLIKEIRVPECAVTE

SEQ ID NO: 4 - protein, 418 aa, E. coli
QSEPELKLESVVIVSRHGVRAPTKATQLMQDVTPDAWPTWPVKLGWLTPRGGELIAYLGHYQRQRLVADGLLAKKGCPQSGQVAIIADVDERTRKTGEAFAAGLAPDCAITVHTQADTSSPDPLFNPLKTGVCQLDNANVTDAILSRAGGSIADFTGHRQTAFRELERVLNFPQSNLCLKREKQDESCSLTQALPSELKVSADNVSLTGAVSLASMLTEIFLLQQAQGMPEPGWGRITDSHQWNTLLSLHNAQFYLLQRTPEVARSRATPLLDLIMAALTPHPPQKQAYGVTLPTSVLFIAGHDTNLANLGGALELNWTLPGQPDNTPPGGELVFERWRRLSDNSQWIQVSLVFQTLQQMRDKTPLSLNTPPGEVKLTLAGCEERNAQGMCSLAGFTQIVNEARIPACSLRSHHHHHH

SEQ ID NO: 5 - protein, 433 aa, Buttiauxella sp.
MQTFGAFLVSFLAASGLAAANDTPASGYQVEKVVILSRHGVRAPTKMTQTMRDVTPNTWPEWPVKLGYITPRGEHLISLMGGFYRQKFQQQGILSQGSCPTPNSIYVWTDVAQRTLKTGEAFLAGLAPQCGLTIHHQQNLEKADPLFHPVKAGICSMDKTQVQQAVEKEAQTPIDNLNQHYIPSLALMNTTLNFSKSPWCQKHSADKSCDLGLSMPSKLSIKDNGNEVSLDGAIGLSSTLAEIFLLEYAQGMPQAAWGNIHSEQEWALLLKLHNVYFDLMERTPYIARHKGTPLLQAISNALNPNATESKLPDISPDNKILFIAGHDTNIANIAGMLNMRWTLPGQPDNTPPGGALVFERLADKSGKQYVSVSMVYQTLEQLRSQTPLSLNQPAGSVQLKIPGCNDQTAEGYCPLSTFTRVVSQSVEPGCQLQ

SEQ ID NO: 6 - protein, 150 aa, Buttiauxella sp.
RHGVRAPTKMTQTMRDVTPNTWPEWPVKLGYITPRGEQLIRVMGGFYREKFQQQGILSQENCPAPNSVYVWADVDQRTLKTGEAFLAGLAPQCGLTIHHQQNLEKADPLFHPVKAGICSMEKNQIQQAVEKEAQNPIDNLNQHYLPSLAL

SEQ ID NO: 7 - protein, 150 aa, Citrobacter braakii
RHGVRAPTKFTPIMKDVTPDQWPQWDVPLGWLTPRGGELVSELGQYQRLWFTSKGLLNNQTCPSPGQVAVIADTDQRTRKTGEAFLAGLAPKCQIQVHYQKDEEKNDPLFNPVKMGKCSFNTLKVKNAILERAGGNIELYTQRYQSSFRT

SEQ ID NO: 8 - protein, 150 aa, Citrobacter freundii
RHGVRAPTKFTPIMKDVTPDQWPQWDVPLGWLTPRGGELVSELGQYQRLWFTSKGLLNNQTCPSPGQVAVIADTDQRTRKTGEAFLAGLAPKCQIQVHYQKDEEKTDPLFNPVKMGTCSFNTLKVKNAILERAGGNIELYTQRYQSSFRT

SEQ ID NO: 9 - protein, 150 aa, Hafnia alvei
RHGVRAPTKMTQTMRDVTPHQWPEWPVKLGYITPRGEHLISLMGGFYRERFQQQGLLPKDNCPTPDAVYVWADVDQRTRKTGEAFLAGLAPQCDLAIHHQQNTQQADPLFHPVKAGICSMDKSQVHAAVEKQAGTPIETLNQRYQASLAL

SEQ ID NO: 10 - protein, 10 aa, Artificial Sequence
  Synthetic
YQVEKVVILS

SEQ ID NO: 11 - protein, 8 aa, Artificial Sequence
  Synthetic
LKTGEAFL

SEQ ID NO: 12 - protein, 13 aa, Artificial Sequence
  Synthetic
LTIHHQQNLEKAD

SEQ ID NO: 13 - protein, 13 aa, Artificial Sequence
  Synthetic
QQAVEKEAQTPID

SEQ ID NO: 14 - protein, 14 aa, Artificial Sequence
  Synthetic
NALNPNATESKLPD

SEQ ID NO: 15 - protein, 8 aa, Artificial Sequence
  Synthetic
LKIPGCND

SEQ ID NO: 16 - protein, 10 aa, Artificial Sequence
  Synthetic
YQVEKVVILS

SEQ ID NO: 17 - protein, 8 aa, Artificial Sequence
  Synthetic
LKTGEAFL

SEQ ID NO: 18 - protein, 13 aa, Artificial Sequence
  Synthetic
LTIHHQQNLEKAD

SEQ ID NO: 19 - protein, 19 aa, Artificial Sequence
  Synthetic
MDKTQVQQAVEKEAQTPID

SEQ ID NO: 20 - protein, 19 aa, Artificial Sequence
  Synthetic
MDKTQVQQAVEKEAQTPID

SEQ ID NO: 21 - protein, 13 aa, Artificial Sequence
  Synthetic
LSIKDNGNEVSLD

SEQ ID NO: 22 - protein, 14 aa, Artificial Sequence
  Synthetic
NALNPNATESKLPD

SEQ ID NO: 23 - protein, 8 aa, Artificial Sequence
  Synthetic
LKIPGCND

SEQ ID NO: 24 - protein, 16 aa, Artificial Sequence
  Synthetic
ADPLFHPVKAGICSMD

SEQ ID NO: 25 - protein, 13 aa, Artificial Sequence
  Synthetic
MDKTQVQQAVEKE

SEQ ID NO: 26 - protein, 12 aa, Artificial Sequence
  Synthetic
PDNKILFIAGHD

SEQ ID NO: 27 - protein, 19 aa, Artificial Sequence
  Synthetic
NDTPASGYQVEKVVILSRH

SEQ ID NO: 28 - protein, 16 aa, Artificial Sequence
  Synthetic
ADPLFHPVKAGICSMD

SEQ ID NO: 29 - protein, 13 aa, Artificial Sequence
  Synthetic
MDKTQVQQAVEKE

SEQ ID NO: 30 - protein, 15 aa, Artificial Sequence
  Synthetic
CDLGLSMPSKLSIKD

SEQ ID NO: 31 - protein, 15 aa, Artificial Sequence
  Synthetic
CDLGLSMPSKLSIKD

SEQ ID NO: 32 - protein, 24 aa, Artificial Sequence
  Synthetic
RHKGTPLLQAISNALNPNATESKL

SEQ ID NO: 33 - protein, 12 aa, Artificial Sequence
  Synthetic
PDNKILFIAGHD

SEQ ID NO: 34 - protein, 24 aa, Artificial Sequence
  Synthetic
RSQTPLSLNQPAGSVQLKIPGCND

SEQ ID NO: 35 - protein, 158 aa, E. coli
QSEPELKLESVVIVSRHGVRAPTKATQLMQDVTPDAWPTWPVKLGWLTPRGGELIAYLGHYQRQRLVADGLLAKKGCPQSGQVAIIADVDERTRKTGEAFAAGLAPDCAITVHTQADTSSPDPLFNPLKTGVCQLDNANVTDAILSRAGGSIADFTGH

SEQ ID NO: 36 - protein, 180 aa, Artificial Sequence
  Synthetic
MSTFIIRLLFFSLLCGSFSIHAEEQNGMKLERVVIVSRHGVRAPTKFTPIMKNVTPDQWPQWDVPLGWLTPRGGELVSELGQYQRLWFTSKGLLNNQTCPSPGQVAVIADTDQRTRKTGEAFLAGLAPKCQIQVHYQKDEEKNDPLFNPVKMGKCSFNTLQVKNAILERAGGNIELYTQR

SEQ ID NO: 37 - protein, 180 aa, Buttiauxella sp.
MQTFGAFLVSFLAASGLAAANDTPASGYQVEKVVILSRHGVRAPTKMTQTMRDVTPNTWPEWPVKLGYITPRGEHLISLMGGFYRQKFQQQGILSQGSCPTPNSIYVWTDVAQRTLKTGEAFLAGLAPQCGLTIHHQQNLEKADPLFHPVKAGICSMDKTQVQQAVEKEAQTPIDNLNQH

SEQ ID NO: 38 - protein, 150 aa, Artificial Sequence
  Synthetic
RHGVRAPTKFTPIMKNVTPDQWPQWDVPLGWLTPRGGELVSELGQYQRLWFTSKGLLNNQTCPSPGQVAVIADTDQRTRKTGEAFLAGLAPKCQIQVHYQKDEEKNDPLFNPVKMGKCSFNTLQVKNAILERAGGNIELYTQRYQSSFRT

SEQ ID NO: 39 - protein, 150 aa, E. coli
RHGVRAPTKATQLMQDVTPDAWPTWPVKLGWLTPRGGELIAYLGHYQRQRLVADGLLAKKGCPQSGQVAIIADVDERTRKTGEAFAAGLAPDCAITVHTQADTSSPDPLFNPLKTGVCQLDNANVTDAILSRAGGSIADFTGHRQTAFRE

SEQ ID NO: 40 - protein, 150 aa, Buttiauxella sp.
RHGVRAPTKMTQTMRDVTPNTWPEWPVKLGYITPRGEHLISLMGGFYRQKFQQQGILSQGSCPTPNSIYVWTDVAQRTLKTGEAFLAGLAPQCGLTIHHQQNLEKADPLFHPVKAGICSMDKTQVQQAVEKEAQTPIDNLNQHYIPSLAL

(19) United States
(12) Patent Application Publication
(10) Pub. No.: US 2016/0289653 A1
(43) Pub. Date: Oct. 6, 2016

(54) Stable Enzymes by Glycation Reduction

(71) Applicant: DANISCO US INC., Palo Alto, CA (US)

(72) Inventors: Mark S. Gebert, Pacifica, CA (US); Soren Dalsgaard, Brabrand (DK); Mariliz Ortiz Johnson, San Mateo, CA (US); Adam L. Garske, Palo Alto, CA (US); Douglas A. Dale, Pacifica, CA (US)

(73) Assignee: Danisco US Inc., Palo Alto, CA (US)

(21) Appl. No.: 15/035,666

(22) PCT Filed: Nov. 14, 2014
(86) PCT No.: PCT/US2014/065612

§ 371 Date: May 10, 2016

Related U.S. Application Data
(60) Provisional application No. 61/904,378, filed on Nov. 14, 2013.
(60) Provisional application No. 61/918,396, filed on Dec. 19, 2013.

(51) Int. Cl. C12N 9/16

(52) U.S. Cl. CPC C12N 9/16; C12Y 301/00

(57) ABSTRACT

The present teachings provide enzymes with improved stability arising from reduced glycation. Glycation reduction can be achieved by a variety of means, including protein engineering, heating to remove background ground starch hydrolytic activity, diafiltration afiltration, and various formulation approaches including non-inclusion of problematic sugars, and inclusion of resistant starches.

### CROSS REFERENCE TO RELATED APPLICATIONS

This application claims benefit of priority from U.S. Provisional Patent Application Nos. U.S. Ser. No. 61/904,378, filed 14 Nov. 2013 and U.S. Ser. No. 61/918,396, filed 19 Dec. 2013, the contents of which are incorporated herein by reference in their entirety.

### FIELD OF THE INVENTION

This disclosure is directed towards improved enzymes, and enzyme formulations.

### BACKGROUND OF THE INVENTION

The use of active agents, such as enzymes, in detergents, foods, and animal feed has become a common practice. There is an ongoing need in these industries for stable enzyme granules that maintain activity after being subjected to harsh conditions.

Approaches to avoid the problem of irreversibly inactivating enzymes or reducing the activity of the enzyme in industrial processes include identifying new sources of an enzyme (e.g. the identification of a known enzyme in an extreme thermophile microorganism) or identifying means to stabilize known enzymes. Klibanov, 1983, (Stabilization of Enzymes against Thermal Inactivation, Advances in Applied Microbiology, volume 29, page 1-28) discloses that there are three basic means for stabilizing enzymes: (1) immobilization, (2) chemical modification and (3) inclusion of additives. While previous formulation approaches have made some progress in this area (see for example WO9854980, WO9739116, WO2007044968), and EP1996028) the present teachings make an additional advance in overcoming some of these problems by recognizing that for certain enzymes reduced glycation can improve enzyme stability.

For ease of reference we have described elements of the present teachings under one or more headings. It is to be noted that the teachings under each of the headings also apply to the teachings under the other headings. For example, each of the stated embodiments and aspects concerning the use of the present teachings is equally an embodiment or aspect concerning the method of the present teachings or the composition of the present teachings. Likewise, each of the stated embodiments and aspects concerning the method or use of the present teachings is equally an embodiment or aspect concerning the composition of the present teachings.

All patents, patent applications, publications, documents, and articles cited herein are all incorporated herein by reference in their entireties.

### SUMMARY OF THE INVENTION

The present teachings provide improved enzymes, formulated enzymes, and methods of making them.

### BRIEF DESCRIPTION OF THE FIGURES

FIGS. 1A-B depict illustrative data according to the present teachings.

FIGS. 2A-D depict illustrative data according to the present teachings.

FIG. 3 depicts illustrative data according to the present teachings.

FIG. 4 depicts illustrative data according to the present teachings.

FIG. 5 depicts illustrative data according to the present teachings.

FIG. 6 depicts illustrative data according to the present teachings.

FIGS. 7A-B depict illustrative data according to the present teachings.

FIG. 8 depicts illustrative data according to the present teachings.

FIG. 9 depicts illustrative data according to the present teachings.

FIG. 10 depicts illustrative data according to the present teachings.

FIG. 11 depicts illustrative data according to the present teachings.

FIG. 12 depicts illustrative data according to the present teachings.

FIG. 13 depicts illustrative data according to the present teachings.

FIG. 14 depicts illustrative data according to the present teachings.

FIGS. 15A-B depict illustrative data according to the present teachings.

FIGS. 16A-B depict illustrative data according to the present teachings.

FIG. 17 depicts illustrative data according to the present teachings.

FIGS. 18A-C depict illustrative data according to the present teachings.

FIG. 19 depicts illustrative data according to the present teachings.

FIG. 20 depicts illustrative data according to the present teachings.

### DETAILED DESCRIPTION

The practice of the present teachings will employ, unless otherwise indicated, conventional techniques of molecular biology (including recombinant techniques), microbiology, cell biology, biochemistry, and animal feed pelleting, which are within the skill of the art. Such techniques are explained fully in the literature, for example, Molecular Cloning: A Laboratory Manual, second edition (Sambrook et al., 1989); Oligonucleotide Synthesis (M. J. Gait, ed., 1984; Current Protocols in Molecular Biology (F. M. Ausubel et al., eds., 1994); PCR: The Polymerase Chain Reaction (Mullis et al., eds., 1994); Gene Transfer and Expression: A Laboratory Manual (Kriegler, 1990), and Fairfield, D. 1994. Chapter 10, Pelleting Cost Center. In Feed Manufacturing Technology IV. (McEllhiney, editor), American Feed Industry Association, Arlington, Va., pp. 110-139.

Unless defined otherwise herein, all technical and scientific terms used herein have the same meaning as commonly understood by one of ordinary skill in the art to which the present teachings belong. Singleton, et al., Dictionary of Microbiology and Molecular Biology, second ed., John Wiley and Sons, N.Y. (1994), and Hale & Markham, The Harper Collins Dictionary of Biology, Harper Perennial, N.Y. (1991) provide one of skill with a general dictionary of many of the terms used in this invention. Any methods and materials similar or equivalent to those described herein can be used in the practice or testing of the present teachings.

Numeric ranges provided herein are inclusive of the numbers defining the range.

### Definitions

As used herein, the term “granule” refers to a particle which contains a core, an active agent, and at least one coating layer.

As used herein, the term “core” refers to the inner nucleus of a granule. The cores of the present teachings may be produced by a variety of fabrication techniques including: rotary atomization, wet granulation, dry granulation, spray drying, disc granulation, extrusion, pan coating, spheronization, drum granulation, fluid-bed agglomeration, high-shear granulation, fluid-bed spray coating, crystallization, precipitation, emulsion gelation, spinning disc atomization and other casting approaches, and prill processes. Such processes are known in the art and are described in U.S. Pat. No. 4,689,297 and U.S. Pat. No. 5,324,649 (fluid bed processing); EP656058B1 and U.S. Pat. No. 454,332 (extrusion process); U.S. Pat. No. 6,248,706 (granulation, high-shear); and EP804532B1 and U.S. Pat. No. 6,534,466 (combination processes utilizing a fluid bed core and mixer coating).

The core may include the enzyme, which may or may not be coated around a seed. Suitable cores for use in the present teachings are preferably a hydratable or porous material (i.e., a material which is dispersible or soluble in water) that is a feed grade material. The core material can either disperse in water (disintegrate when hydrated) or solubilize in water by going into a true aqueous solution. Clays (for example, the phyllosilicates bentonite, kaolin, montmorillonite, hectorite, saponite, beidellite, attapulgite, and stevensite), silicates, such as sand (sodium silicate), nonpareils and agglomerated potato starch or flour, or other starch granule sources such as wheat and corn cobs are considered dispersible. (Nonpareils are spherical particles made of a seed crystal that has been built onto and rounded into a spherical shape by binding layers of powder and solute to the seed crystal in a rotating spherical container. Nonpareils are typically made from a combination of a sugar such as sucrose, and a powder such as cornstarch.) In one embodiment of the present teachings the core comprises a sodium chloride or sodium sulfate crystal, also referred to as a seed, or other inorganic salt crystal. In another embodiment of the present teachings, the core comprises a sucrose crystal seed. Particles composed of inorganic salts and/or sugars and/or small organic molecules may be used as the cores of the present teachings. Suitable water soluble ingredients for incorporation into cores include: inorganic salts such as sodium chloride, ammonium sulfate, sodium sulfate, magnesium sulfate, zinc sulfate; or urea, citric acid, sugars such as sucrose, lactose and the like. Cores of the present teachings may further comprise one or more of the following: additional active agents, feed or food grade polymers, fillers, plasticizers, fibrous materials, extenders and other compounds known to be used in cores. Suitable polymers include polyvinyl alcohol (PVA), including partially and fully hydrolyzed PVA, polyethylene glycol, polyethylene oxide, polyvinyl pyrrolidine, and carbohydrate polymers (such as starch, amylose, amylopectin, alpha and beta-glucans, pectin, glycogen), including mixtures and derivatives thereof. Suitable fillers useful in the cores include inert materials used to add bulk and reduce cost, or used for the purpose of adjusting the intended enzyme activity in the finished granule. Examples of such fillers include, but are not limited to, water soluble agents such as salts, sugars and water dispersible agents such as clays, talc, silicates, cellulose and starches, and cellulose and starch derivatives. Suitable plasticizers useful in the cores of the present teachings are low molecular weight organic compounds and are highly specific to the polymer being plasticized. Examples include, but are not limited to, sugars (such as, glucose, fructose and sucrose), sugar alcohols (such as, sorbitol, xylitol and maltitol and other glycols), polar low molecular weight organic compounds, such as urea, or other known plasticizers such as water or feed grade plasticizers. Suitable fibrous materials useful in the cores of the present teachings include, but are not limited to: cellulose, and cellulose derivatives such as HPMC (hydroxy-propyl-methyl cellulose), CMC (carboxy-methyl cellulose), HEC (hydroxy-ethyl cellulose). In another embodiment particularly suitable for household cleaning applications, the core comprises a water-soluble or dispersible sugar or salt crystal or a non pareil. Those skilled in the art will recognize that, for feed and food applications, the cores (and any polymers, fillers, plasticizers, fibrous materials, and extenders), are acceptable for food and/or feed applications. For household cleaning applications, such a restriction need not apply.

Any enzyme susceptible to problematic glycation may be used according to the present teachings, as readily assessed by one of skill in the art, and a nonlimiting list of enzymes may include phytases, xylanases, β-glucanases, phosphatases, proteases, amylases (alpha or beta or glucoamylases) cellulases, lipases, cutinases, oxidases, transferases, reductases, hemicellulases, mannanases, esterases, isomerases, pectinases, lactases, peroxidases, laccases, other redox enzymes and mixtures thereof. Particularly preferred enzymes may include a xylanase from Trichoderma reesei and a variant xylanase from Trichoderma reesei, both available from DuPont Industrial Biosciences or the inherently thermostable xylanase described in EP1222256B1, as well as other xylanases from Aspergillus niger, Aspergillus kawachii, Aspergillus tubigensis, Bacillus circulans, Bacillus pumilus, Bacillus subtilis, Neocallimastix patriciarum,Penicillium species, Streptomyces lividans, Streptomyces thermoviolaceus, Thermomonospora fusca, Trichoderma harzianum, Trichoderma reesei, Trichoderma viride. Additional enzymes may include phytases, such as for example Finase L®, a phytase from Aspergillus sp., available from AB Enzymes, Darmstadt, Germany; Phyzyme™ XP, a phytase from E. Coli, available from DuPont Nutrition and Health, and other phytases from, for example, the following organisms: Trichoderma, Penicillium, Fusarium, Buttiauxella, Citrobacter, Enterobacter, Penicillium, Humicola, Bacillus, and Peniophora, as well as those phytases described in U.S. patent applications 61/595,923 and 61/595,941, both filed Feb. 12, 2012. An example of a potential cellullase is Multifect® BGL, a cellulase (beta glucanase), available from DuPont Industrial Biosciences and other cellulases from species such as Aspergillus, Trichoderma, Penicillium, Humicola, Bacillus, Cellulomonas, Penicillium, Thermomonospore, Clostridium, and Hypocrea. The cellulases and endoglucanases described in US20060193897A1 also may be used. Commercially available cellulases that find use in the present include, but are not limited to CELLUZYME®, CAREZYME® (Novozymes), and KAC-500(B)™ (Kao Corporation). Amylases may be, for example, from species such as Aspergillus, Trichoderma, Penicillium, Bacillus, for instance, B. subtilis, B. stearothermophilus, B. lentus, B. licheniformis, B. coagulans, and B. amyloliquefaciens. Potentially suitable fungal amylases are derived from Aspergillus, such as A. oryzae and A. niger. Commercially available amylases that may find use in the present invention include, but are not limited to DURAMYL®, TERMAMYL®, FUNGAMYL®, STAINZYME®, STAINZYME PLUS®, STAINZYME ULTRA®, and BAN™ (Novozymes), as well as POWERASE™, RAPIDASE® and MAXAMYL® P (Genencor). Proteases may be from Bacillus amyloliquefaciens, Bacillus lentus, Bacillus subtilis, Bacillus licheniformis, and Aspergillus and Trichoderma species. Phytases, xylanases, phosphatases, proteases, amylases, esterases, redox enzymes, lipases, transferases, cellulases, and β-glucanases are enzymes frequently used for inclusion in animal feed and may find application in the present teachings. Suitable proteases may include those of animal, vegetable or microbial origin. In some embodiments, microbial proteases may be used. In some embodiments, chemically or genetically modified mutants may be included. In some embodiments, the protease may be a serine protease, preferably an alkaline microbial protease or a trypsin-like protease. Examples of alkaline proteases may include subtilisins, especially those derived from Bacillus (e.g., subtilisin, lentus, amyloliquefaciens, subtilisin Carlsberg, subtilisin 309, subtilisin 147 and subtilisin 168). Additional examples may include those mutant proteases described in U.S. Pat. Nos. RE 34,606, 5,955,340, 5,700,676, 6,312,936, and 6,482,628, all of which are incorporated herein by reference. Additional protease examples may include, but are not limited to trypsin (e.g., of porcine or bovine origin), and the Fusarium protease described in WO 89/06270. In some embodiments, commercially available protease enzymes that may find use in the present invention include, but are not limited to MAXATASE®, MAXACAL™, MAXAPEM™, OPTICLEAN®, OPTIMASE®, PROPERASE®, PURAFECT®, PURAFECT® OXP, PURAMAX™, EXCELLASE™, and PURAFAST™ (Genencor); ALCALASE®, SAVINASE®, PRIMASE®, DURAZYM™, POLARZYME®, OVOZYME®, KANNASE®, LIQUANASE®, NEUTRASE®, RELASE® and ESPERASE® (Novozymes); BLAP™ and BLAP™ variants (Henkel Kommanditgesellschaft auf Aktien, Duesseldorf, Germany), and KAP (B. alkalophilus subtilisin; Kao Corp., Tokyo, Japan). Various proteases are described in WO95/23221, WO 92/21760, WO 09/149200, WO 09/149144, WO 09/149145, WO 11/072099, WO 10/056640, WO 10/056653, WO 11/140364, WO 12/151534, U.S. Pat. Publ. No. 2008/0090747, and U.S. Pat. Nos. 5,801,039, 5,340,735, 5,500,364, 5,855,625, U.S. Pat. Nos. RE 34,606, 5,955,340, 5,700,676, 6,312,936, and 6,482,628, and various other patents. In some further embodiments, metalloproteases may find use in the present invention, including but not limited to the neutral metalloprotease described in WO 07/044993. Potential enzymes suitable for inclusion into detergents for household care applications are similar, particularly proteases, amylases, lipases, hemicellulases, redox enzymes, peroxidases, mannanases, pectinases, polyesterases, transferases, and cellulases. In some aspects of the present teachings, the enzymes may be selected from phytases, xylanases, beta glucanases, amylases, proteases, lipases, esterases, and mixtures thereof. In one embodiment of the present invention, two enzymes are provided in the granule, a xylanase and a beta-glucanase. The enzymes may be mixed together or applied to the granule separately. In another embodiment, three enzymes are provided in the granule, namely beta-glucanase, xylanase and phytase. In some embodiments, the granule can be in a composition with other granules, builders, surfactants, and other materials. The above enzyme lists are examples only and are not meant to be exclusive. Any enzyme may be used in the present invention that is susceptible to problematic glycation, including wild type, recombinant and variant enzymes of bacterial, fungal, yeast, plant, insect and animal sources, and acid, neutral or alkaline enzymes.

As used herein, the term “enzyme matrix” refers to recovered enzyme arising from a fermentation, and may include additional components as processing aids including for example binders (e.g. PVA), anti-foam agents, surfactants (e.g. lutensol), starch, and sugars.

The term “coating layer” and “layer” as used herein are interchangeably. The first coating layer generally encapsulates the core in order to form a substantially continuous layer so that the core surface has few or no uncoated areas. Subsequent coating layers can encapsulate the growing granule to form one or more additional substantially continuous layer(s). Accordingly, as used herein, an “additional layer” refers to one or more coatings applied to a granule, and can contain any of a variety of materials readily available to one of routine skill in the art, including those materials described under “cores”, as well as may be found in the relevant arts, including U.S. Pat. No. 7,018,821, U.S. Pat. No. 8,076,113, U.S. Pat. No. 4,106,991, U.S. Pat. No. 4,689,297, and U.S. Pat. No. 4,740,469.

As used herein, the term “resistant starch” refers to a starch that has been modified to reduce the production of glucose when treated with a glucoamylase. Specifically, a starch as used herein will be deemed “resistant starch” if less than 0.2 mM glucose is produced when 2% w/w of the starch of interest is incubated with Trichoderma reesei wild type glucoamylase for 40 minutes at 25 C in 50 mM NaOAC (pH 4.5), by measuring total reducing ends with the BCA method using a glucose calibration curve.

As used herein, the term “enzyme susceptible to problematic glycation” refers to an enzyme which when subjected to the following assay shows at least a 20% reduction in activity relative to the control. An enzyme solution is obtained, 1% w/w glucose is added to it, the resulting mixture is lyophilized, and the resulting dry solid is exposed to 50 C/70% RH for 5 days, redissolved, and the activity of this sample is measured and compared to a control sample exposed to 50 C/to % RH but without the added glucose. If the enzyme with glucose added loses more than 20% activity than the control, it is “susceptible to problematic glycation.” In some embodiments, at least a 30%, at least a 40%, at least a 50%, at least a 60%, at least a 70%, at least an 80%, or at least a 90% reduction is produced.

As used herein, the term “problematic sugar” generally refers to glucose, fructose, and sucrose.

As used herein, the term “glycatable amino acid residue” refers to an amino acid in a protein that is susceptible of undergoing covalent addition of a sugar. Examples include lysine and arginine, though it will be appreciated that other non-naturally occurring amino acids, if found in a protein and susceptible of undergoing covalent addition of a sugar, would fall within the scope of the present teachings.

As used herein, the term “one diavolume” equals the amount of filtrate recovered in comparison to an equal volume of retentate held at constant volume while adding fresh water or buffer during a process of continuous diafiltration or dialysis.

As used herein, the term “Percent sequence identity” means that a variant has at least a certain percentage of amino acid residues identical to a protein of interest, when aligned using the CLUSTAL W algorithm with default parameters. See Thompson et al. (1994) Nucleic Acids Res. 22:4673-4680. Default parameters for the CLUSTAL W algorithm are:

| | |
|---|---|
| Gap opening penalty: | 10.0 |
| Gap extension penalty: | 0.05 |
| Protein weight matrix: | BLOSUM series |
| DNA weight matrix: | IUB |
| Delay divergent sequences %: | 40 |
| Gap separation distance: | 8 |
| DNA transitions weight: | 0.50 |
| List hydrophilic residues: | GPSNDQEKR |
| Use negative matrix: | OFF |
| Toggle Residue specific penalties: | ON |
| Toggle hydrophilic penalties: | ON |
| Toggle end gap separation penalty | OFF. |

Deletions are counted as non-identical residues, compared to a reference sequence. Deletions occurring at either terminus are included. For example, a variant with six amino acid deletions of the C-terminus of a mature polypeptide 612 residue molecule would have a percent sequence identity of 99% (606/612 identical residues×100, rounded to the nearest whole number) relative to the mature polypeptide. Such a variant would be encompassed by a variant having “at least 99% sequence identity” to a protein of interest.

### Exemplary Embodiments

In an embodiment illustrative of the present teachings, and enzyme susceptible to problematic glycation is identified. Thereafter, the enzyme is made so as to ensure glycation is reduced relative to an enzyme made without such ensurances, so as to improve enzyme stability relative thereto. Various approaches can be undertaken so as to ensure glycation is reduced, as provided in the present examples and discussed in the included claims, and include diafiltration, engineering out problematic amino acid residues, heating to inactivate problematic enzymes remaining from a fermentation, formulating without problematic sugars, formulating using resistant starches, and combinations thereof.

In some embodiments, the present teachings provide a method of improving enzyme stability comprising; making an enzyme in a fermentation, wherein the fermentation comprises a fermentation medium comprising problematic sugars and hydrolyzable starch, and wherein the enzyme comprises at least one glycatable amino acid residue and is an enzyme susceptible to problematic glycation; recovering the enzyme; and, removing sufficient amounts of the problematic sugars to improve enzyme stability relative to a similarly fermented enzyme lacking the removing.

In some embodiments, the removing comprises diafiltering an ultrafiltrate concentrate to remove the problematic sugars to form an enzyme concentrate. In some embodiments, the diafiltering comprises at least two, at least three, or at least four, diavolumes, such that the sugars content of the enzyme concentrate is reduced to less than 0.1% w/w. In some embodiments, the removing comprises heating the enzyme after the fermentation at 50C for at least 1, 2, 4, 8, or 12 hours to inactivate any starch-hydrolyzing enzymes present. In some embodiments, the removing comprises a previous genetic manipulation, wherein at least one active site lysine residue was removed from the polypeptide. In some embodiments, the active lysine residue is K131 and/or K26, and the enzyme is wild type Buttauxella sp. Phytase, or a molecule 90%, 95%, 97%, 98%, or 99% identical thereto. In some embodiments, the active lysine residue is K131 and/or K26, and the enzyme is BP17.

In some embodiments, the present teachings provide an enzyme granule comprising less than 0.1%, 0.08%, 0.06%, 0.04%, or 0.01% w/w of problematic sugars. In some embodiments, the enzyme resides in a layer. In some embodiments, the enzyme resides throughout the granule.

In some embodiments, the present teachings provide an engineered phytase wherein an amino acid residue at position 131 and/or 26 relative to wild-type buttiauxella phytase is not lysine. In some embodiments, the position 131 residue is arginine.

In some embodiments, the present teachings provide a method of improving an enzyme's stability comprising; determining the existence of at least one glycatable amino acid residue in an enzyme; determining whether the enzyme is susceptible to problematic glycation; making the enzyme so as to ensure glycation is reduced relative to an enzyme made without such ensurances, so as to improve enzyme stability relative thereto. In some embodiments, the ensurances comprise mutating at least one lysine residue. In some embodiments, the ensurances comprise treating the enzyme with heat. In some embodiments, the ensurances comprise formulation the enzyme in a granule comprising resistant starch. In some embodiments, the ensurances comprise diafiltering the enzyme. In some embodiments, the ensurances comprises combining various approaches, for example, mutating and heat treatment, or, mutating and formulating with resistant starch, or, mutating and diafiltering, or, heat treatment and diafiltering, or, mutating and diafiltering, or, mutating and heat treatment and diafiltering, or, in other combinations as will be appreciated by one of ordinary skill in the art exercising routine combinatorial logic.

In some embodiments, the present teachings provide an enzyme granule comprising an enzyme susceptible to problematic glycation, and, a resistant starch. In some embodiments, the enzyme granule comprises a) a sodium sulfate seed, b) an enzyme layer comprising the enzyme susceptible to problematic glycation, the resistant starch, sodium phytate, and rapeseed oil, c) a sodium sulfate layer, and, d) an external layer comprising PVA and talc. In some embodiments, the enzyme present in the enzyme granule is a phytase. In some embodiments, the enzyme is wild type Buttiauxella sp. In some embodiments, the enzyme is BP17, or an amino acid sequence 90%, 95%, 97%, or 99% identical to it. In some embodiments, the enzyme is a cellulase. In some embodiments, the enzyme is the cellulase exemplified herein, or an amino acid sequence 90%, 95%, 97%, or 99% identical to it.

The enzymes and compositions of the present teachings will found application of any of a variety of fields, including grain and starch processing (e.g.-amylases and glucoamylases), fabric and household care (e.g.-proteases), biomass processing (e.g.-cellulases), animal feed (e.g.-phytases). These and other application areas and proteins will be appreciated by one of skill in the art, and aren't generally a limitation of the present teachings.

The enzymes of the present teachings can be formulated as solids (eg-enzyme granules), or as liquids (e.g.-liquids containing suitable amounts of for examples sorbitol and salt), or indeed in any form that one of skill in the art would recognize their applicability.

The invention can be further understood by reference to the following examples, which is provided by way of illustration and not meant to be limiting.

### EXAMPLE 1

### Activity Loss Data

To explore the effect of time on phytase stability, a BP17 phytase was expressed and purified to form a UFC.

BP17 is an enzyme variant of a Buttiauxella sp. phytase. The sequence for BP17 (excluding signal peptide) is as follows, and is described in PCT/US2013/0244415.

SEQ ID NO: 1
NDTPASGYQVEKVVILSRHGVRAPTKMTQTMRDVTPNTWPEWPVKLG
YITPRGEHLISLMGGFYRQKFQQQGILSQGSCPTPNSIYVWTDVAQR
TLKTGEAFLAGLAPQCGLTIHHQQNLEKADPLFHPVKAGICSMDKTQ
VQQAVEKEAQTPIDNLNQHYIPSLALMNTTLNFSKSPWCQKHSADKS
CDLGLSMPSKLSIKDNGNEVSLDGAIGLSSTLAEIFLLEYAQGMPQA
AWGNIHSEQEWALLLKLHNVYFDLMERTPYIARHKGTPLLQAISNAL
NPNATESKLPDISPDNKILFIAGHDTNIANIAGMLNMRWTLPGQPDN
TPPGGALVFERLADKSGKQYVSVSMVYQTLEQLRSQTPLSLNQPAGS
VQLKIPGCNDQTAEGYCPLSTFTRVVSQSVEPGCQLQ

The fermentation of proteins including BP17 phytase is described in WO2004035070A1 and U.S. Pat. No. 7,713,725B2. After fermentation, the fermentate is passed through a rotary drum vacuum filtration unit which can be precoated with diatomaceous earth to remove the cells from the fermentate. Next, the cell free fermentate is concentrated by passing it through a 10,000 molecular weight cutoff ultrafiltration membrane removing enough permeate until the final activity is between 30,000 and 80,000 FTU/g.

The recovered fermentation concentrate is called “ultrafiltered concentrate” or UFC and contains the phytase protein along with other proteins, peptides, polysaccharides and sugars produced during the fermentation as byproducts. The UFC is combined with 0.3% Potassium Sorbate and 0.3% Sodium Benzoate to serve as a preservative prior to granulating the material. To make a 2000 gram batch of Axtra Phytase TPT®, 380 g of Phytase UFC having an activity of 74,250 units/g and a solids content of 25.6% is combined with 190 g of water, 70.6 g of sucrose, 152.9 g of corn starch, 17.6 g of rapeseed oil and 11.8 g of sodium phytate and this mixture is sprayed onto 722 g of sodium sulfate seeds which have been charged to a Vector VFC-1 fluid bed granulator. For this first spray coating, the inlet temperature was 60° C., the outlet temperature was 40° C., the spray rate was 11 g/min, the atomization air pressure was 38 psi and the fluidization air velocity was 75 cfm. Next a solution of 870 g of sodium sulfate dissolved in 2475 g of water is sprayed onto the granule using an inlet temperature was 68° C., the outlet temperature was 47° C., the spray rate was 25 g/min, the atomization air pressure was 38 psi and the fluidization air velocity was 75 cfm. The final coating was prepared by dissolving 70.59 g of polyvinyl alcohol (Mowial 5-88®) in 965 g of water with 141 g of talc at 90° C. for 30 minutes. After this solution is allowed to cool back to room temperature, it is sprayed onto the granule using an inlet temperature of 70° C., an outlet temperature of 51° C., a spray rate of 11 g/min, an atomization air pressure of 40 psi and a fluidization air velocity of 75 cfm. Then, 20 gram samples of Axtra Phytase TPT® enzyme granules were placed in a 200 ml closed polyethylene container stored at 4° C., 25° C. and 40° C. for 12 months. At regular time intervals, phytase activity was determined by both the para-nitrophenyl phosphate (pNPP) assay and the molybdenum vanadat (Mo-V) assay following dissolution with0.25 M Sodium Acetate Buffer, pH 5.5. In the pNPP assay, a synthetic substrate, para-nitrophenyl phosphate, is employed. Upon dephosphorylation with phytase and basification of the reaction mixture, the para-nitrophenylate anion is formed, which absorbs at 405 nm. In the Mo-V assay, sodium phytate is used as a substrate. Phytate desphosphorylation is coupled to absorbance at 415 nm upon treatment with an acidic molybdenum-vanadate cocktail. The pNPP assay is a generic phosphatase assay, while the Mo-V assay is specific to phytase

The pNPP Assay can be performed on an automated Konelab® robot as follows. All samples were diluted in 0.25 M acetate pH 5.5 buffer (Dissolve 30.02 g sodium acetate trihydrate (or 18.10 g sodium acetate anhydrous), 1.76 g concentrated acetic acid (=1.677 ml) and 0.147 g calcium chloride dihydrate in 900 ml Milli-Q water. Adjust to pH 5.5 with 4 M acetic acid (22.9 ml concentrated acetic acid in 100 ml Milli-Q water) or 100% acetic acid. Add 1 ml TWEEN 20 and adjust volume to 1000 ml with Milli-Q water.). Once diluted samples were loaded into the Konelab, 7 μl of sample was combined with 84 μl of pNPP substrate solution (4-nitrophenyl phosphate (Npp) di(-tris) salt (FW 461.4) (Sigma N-3254) Weigh 0.09228 g 4-Nitrophenyl phosphate and dissolve in 10 mL Assay Buffer) and allowed to incubate at 30° C. for 5 minutes. Next, 105 μl of 200 mM borate stop solution (Dissolve 6.183 g boric acid in 400 mL MilliQ water. Adjust pH to 10.2 with 1N NaOH. Q.S. to 500 mL with MilliQ water.) is added to the sample and substrate to quench the reaction for 2 minutes. The absorbance at 405 nm is read and converted to activity units of FTU/g through the use of a calibration curve containing phytase standards at 0, 2.5, 13.0 and 27.0 FTU/g.

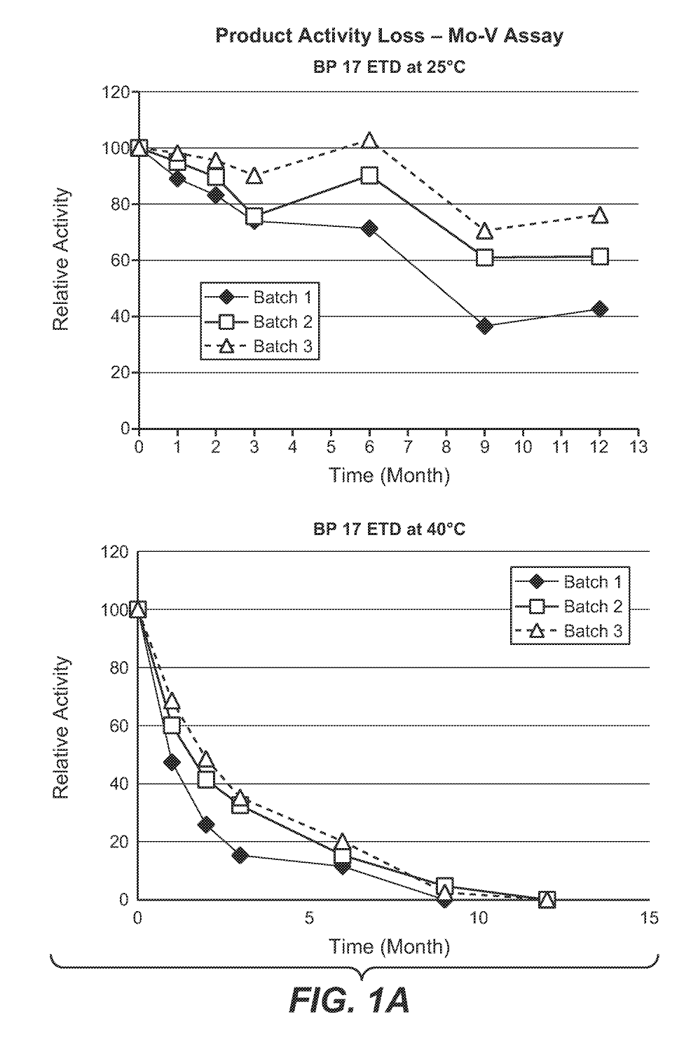

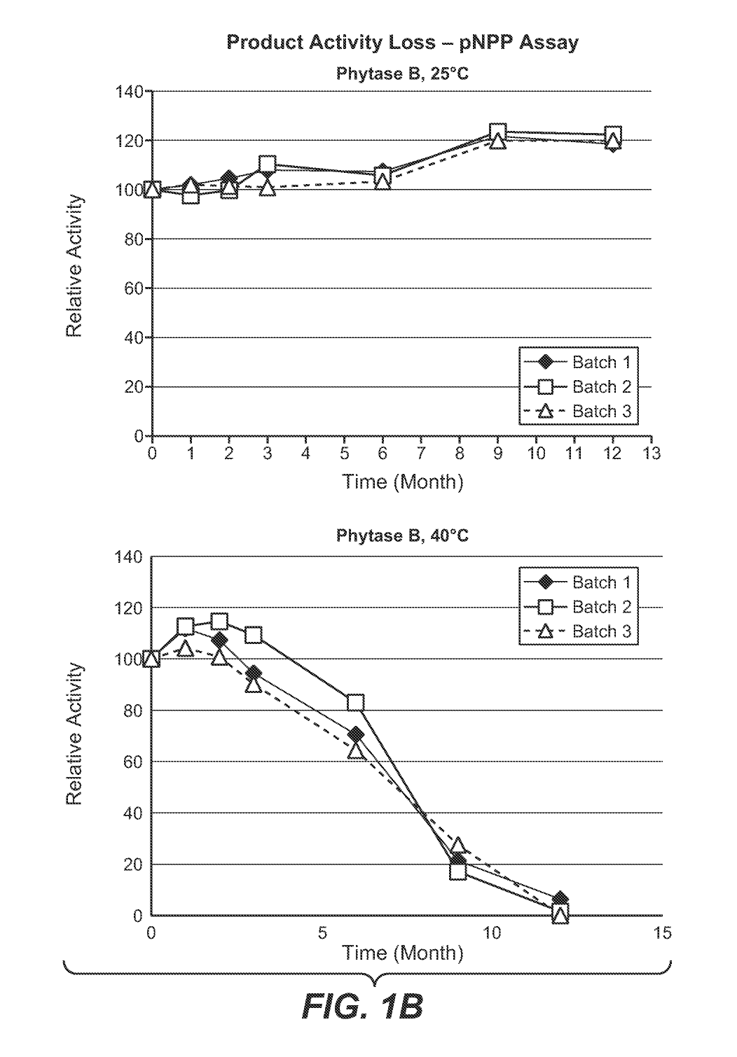

The graphs in FIGS. 1A and 1B summarize the activity loss suffered by TPT Phytase during storage. At 25° C. granule suffer a 30-70% Mo-V activity (sodium phytate substrate) loss over 12 months. No activity is lost in the pNPP assay (synthetic substrate) for the same samples over the same time period. At 40° C. TPT phytase loses 95-100% of its Mo-V activity over 6 months and only 20-40% of its pNPP activity for the same samples over 6 months.

It was hypothesized that the differential loss of activity between the pNPP and Mo-V assay is due to glycation of lysine residues near the phytase active site which could alter substrate specificity towards the larger, natural phytic acid substrate but not for the smaller synthetic para-nitrophenyl phosphate (pNPP) substrate.

### EXAMPLE 2

### Maillard Reaction and Browning

To test the role of glycation, the Maillard browning reaction was monitored visually by depositing a solution BP17 ETD phytase UFC onto a circle of filter paper, drying the enzyme and then placing the enzyme in an environmental chamber at 50° C. and 70% relative humidity for 5 to 12 days.

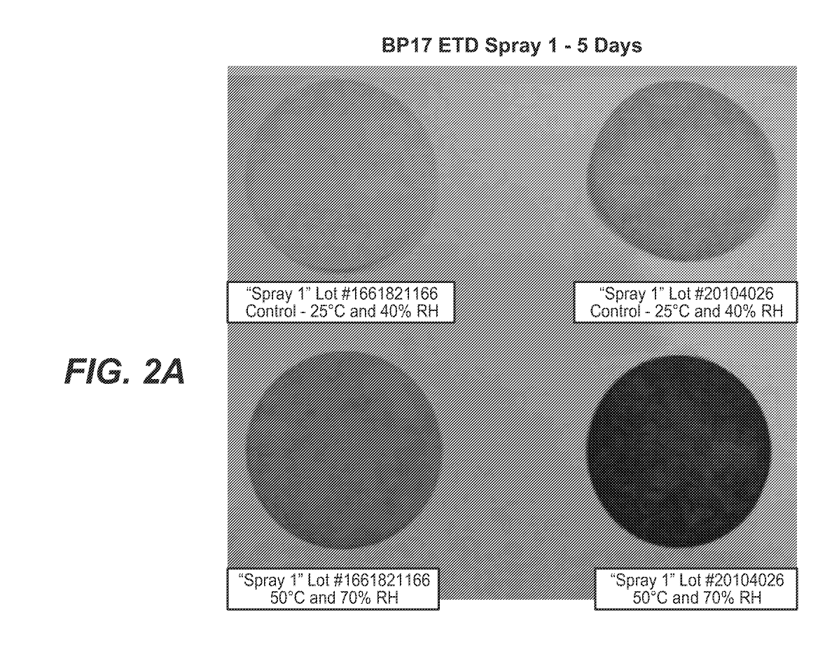

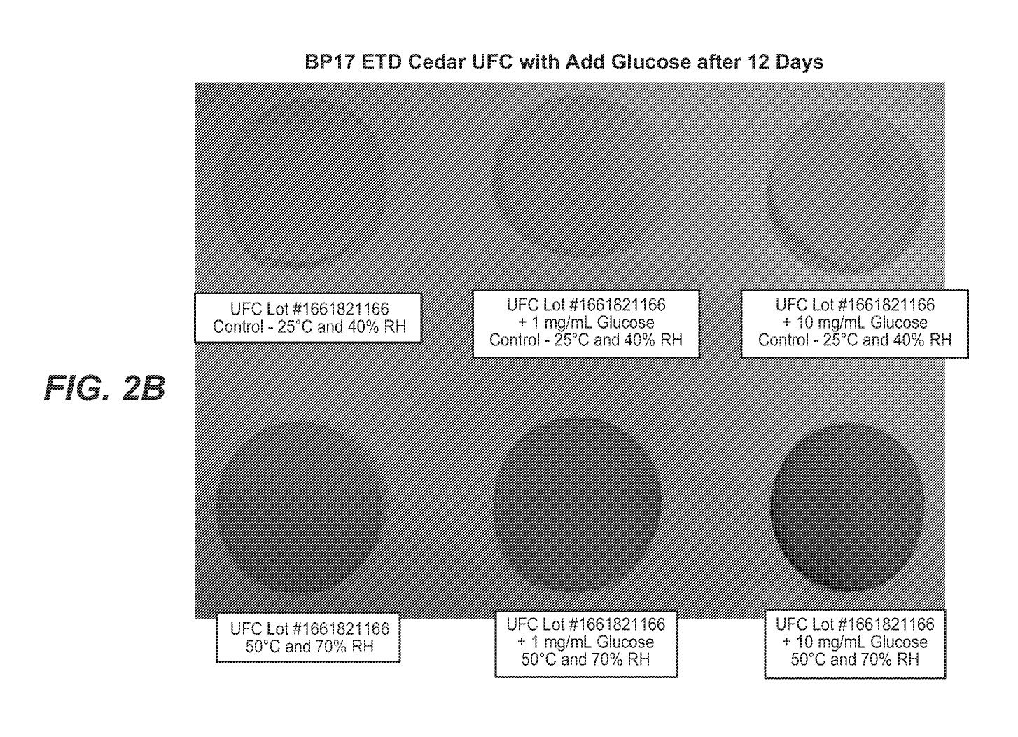

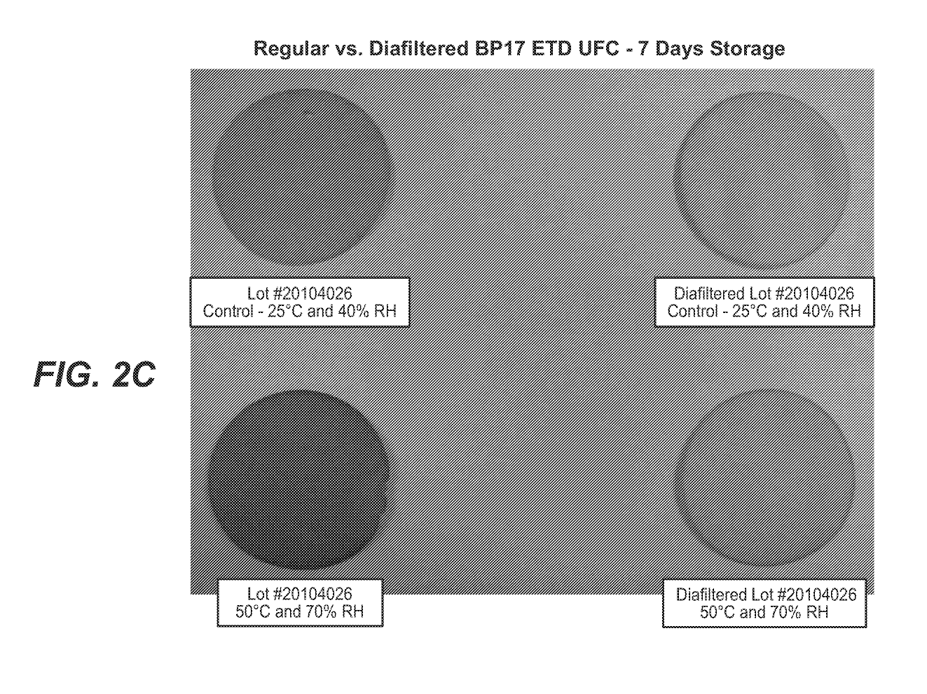

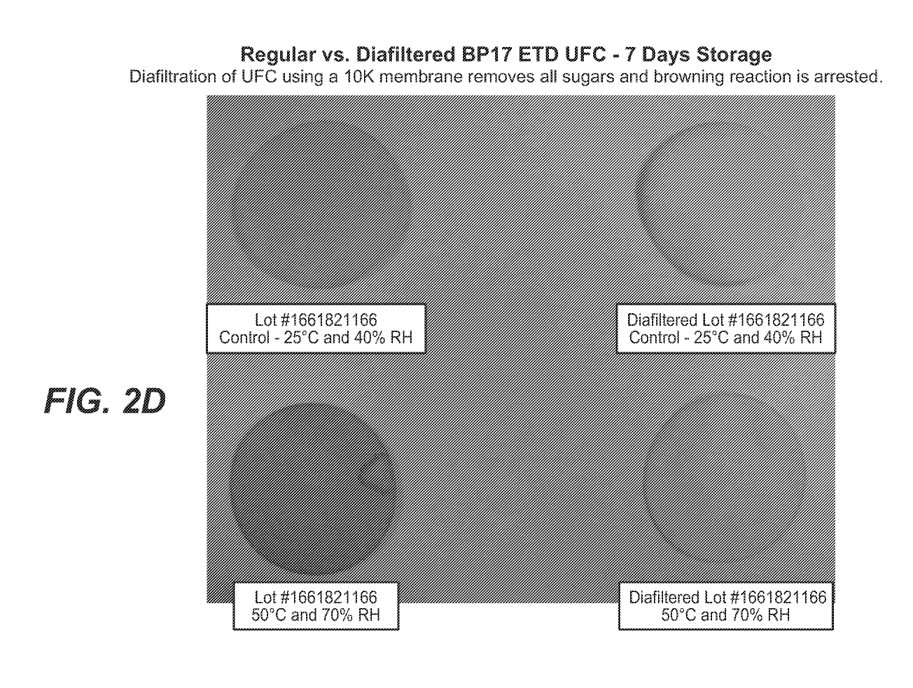

FIG. 2A shows darkening of the dry phytase enzyme spray 1 (composed of 32% enzyme solids, 41% corn starch, 19% sucrose, 3% sodium phytate and 5% rapeseed oil) stored at 50° C. and 70% relative humidity for 5 days for two different lots of enzyme UFC. FIG. 2B shows one lot of phytase UFC with varying levels of added glucose stored at both 25° C./40% RH and 50° C./70% RH for 12 days. It can be seen that increasing levels of glucose, leads to increasing levels of browning.

Diafiltration of BP17 ETD UFC was performed using a 10K molecular weight cutoff membrane to filter away the permeate of the UFC (which contains all of the material less than 10K Daltons). Diafiltration was performed on a Amicon stirred cell (Amicon Model #8400) fitted with a 10K Dalton molecular weight cutoff Polyethersulfone membrane (Sartorius No. 14639—76-----D) on top of a 10K Dalton molecular weight cutoff regenerated cellulose membrane (Millipore Cat. #PLGC07610). By performing dialfiltration to generate 3 times the original volume of the starting UFC in permeate (3 diavolumes), approximately 96% of all reducing sugars including glucose are removed from the UFC, greatly reducing the tendency for this UFC to undergo glycation or the Maillard reaction. The percent of permeable molecules removed during diafiltration can be described by the following equation:

{1−[e^{(V*T)}]⁻¹}×100

where V is the number of diavolumes diafiltered and T is the transmission coefficient through the membrane. When we assume T=1 (100% transmission) and V=3 (3 diavolumes), them the percent permeable molecules diafiltered is (1−e⁻³)×100 or 95%.

FIG. 2C shows the effect of diafiltering BP17 ETD UFC on the browning reaction after 7 days of storage at 50° C. and 70% relative humidity. It can be seen that the UFC shows almost no browning after diafiltering the enzyme UFC 3 diavolumes through a 10K membrane. FIG. 2D shows the effect of diafiltering a second lot of BP17 ETD UFC on the browning reaction after 7 days of storage at 50° C. and 70% relative humidity. It can be seen that the UFC shows almost no browning after diafiltering the enzyme UFC 3 diavolumes through a 10K membrane.

### EXAMPLE 3

### Intrinsic Fluorescence

As proteins undergo the Maillard reaction they will form glycation products, which undergo further chemical reaction to form fluorescent heterocyclic products. The formation of these advanced glycation products can be monitored by measuring the intensity of their fluorescent emission signal. Approximately 0.15 g granule was weighed and mixed with 1.5 ml dilution buffer (100 mM NaOAc, pH 5.5), and allowed to dissolve for 20 min at room temperature. The granules were composed approximately 37% of a 200-300 μm core (a seed of sodium sulfate) coated with an enzyme layer composed of either (A) 2% polyvinyl alcohol (Mowial® 5-88), 0.50% sodium phytate and approximately 4% phytase enzyme solids or, (B) 3% sucrose, 6.5% corn starch, 0.50% sodium phytate and approximately 4% phytase enzyme solids. On top of the enzyme layer is a 40% layer of sodium sulfate followed by a final coating layer composed of 3% polyvinyl alcohol and 6% Talc. The sample was centrifuged at 3000 rpm for 10 min and filtered through 0.45 um filters. Zeba desalt spin columns (10 ml columns) were equilibrated 4×5 ml 20 mM sodium borate buffer at pH 8.5. One milliliter of the sample was added to each column with 0.2 ml of borate buffer to aid elution by centrifugation. The eluted sample was then placed into a microtiter plate and the fluorescence spectrum was monitored using a 335 nm excitation wavelength.

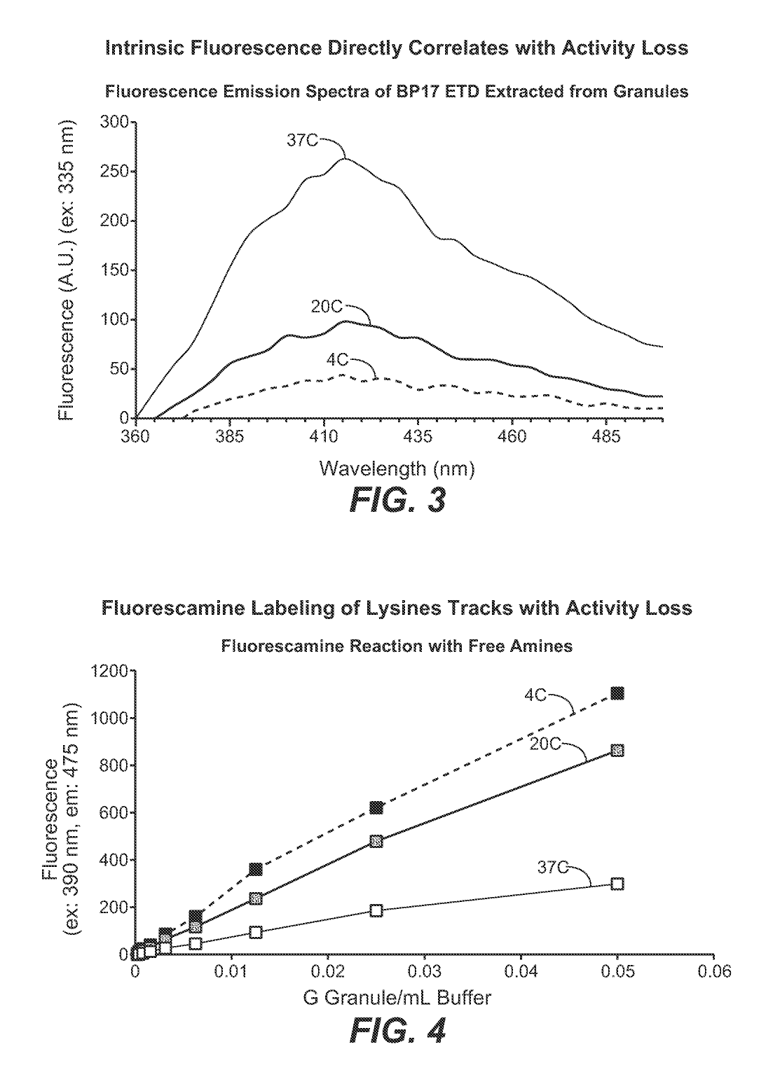

FIG. 3 shows the fluorescence emission spectrum resulting from excitation at 335 nm for phytase extracted from TPT granules stored at 4° C., 25° C. and 37° C. for 12 months. The increase in fluorescence tracks with storage temperature. Since higher storage temperatures accelerate the rate of glycation and higher levels of glycation will lead to higher levels of advanced glycation end products (AGE's) and since the presence of these AGE's is responsible for the intrinsic fluorescence, it is concluded that higher levels of intrinsic fluorescence is also correlated to loss of phytase activity since higher levels of glycation of phytase is directly correlated to loss of phytase activity.

### EXAMPLE 4

### Fluorescamine Assay

Similarly treated samples used in the Intrinsic Fluorescence measurements were used in the Fluorescamine Assay. Fluorescamine was used to directly measure the level of glycated lysine groups in phytase. Fluorescamine reacts with unmodified (non-glycated) lysine groups and becomes fluorescent after reaction.

Fluorescamine (8 mg) was mixed with DMSO (50 ml) to make a solution at a final concentration 0.16 mg/ml. Nine 2-fold serial dilutions of the desalted samples were prepared on a 96 well microtiter plate. Sample quantities of 75 ul were transferred to black fluorescent plates in duplicate. One set was combined with 18 ul of the DMSO/fluorescamine solution and the other was combined with 18 ul of DMSO alone. The solutions are allowed to react at room temperature for 5 minutes on shaker plate with a cover. The samples were excited at 390 nm and emission was monitored at 475 nm. Fluorescent emission values without fluorescamine were subtracted from those with fluorescamine and plotted. FIG. 4 shows that the fluorescence emission intensity increased with concentration of phytase, but decreased with increasing temperature. The latter suggests an increase in glycated lysine groups as storage temperature is increased.

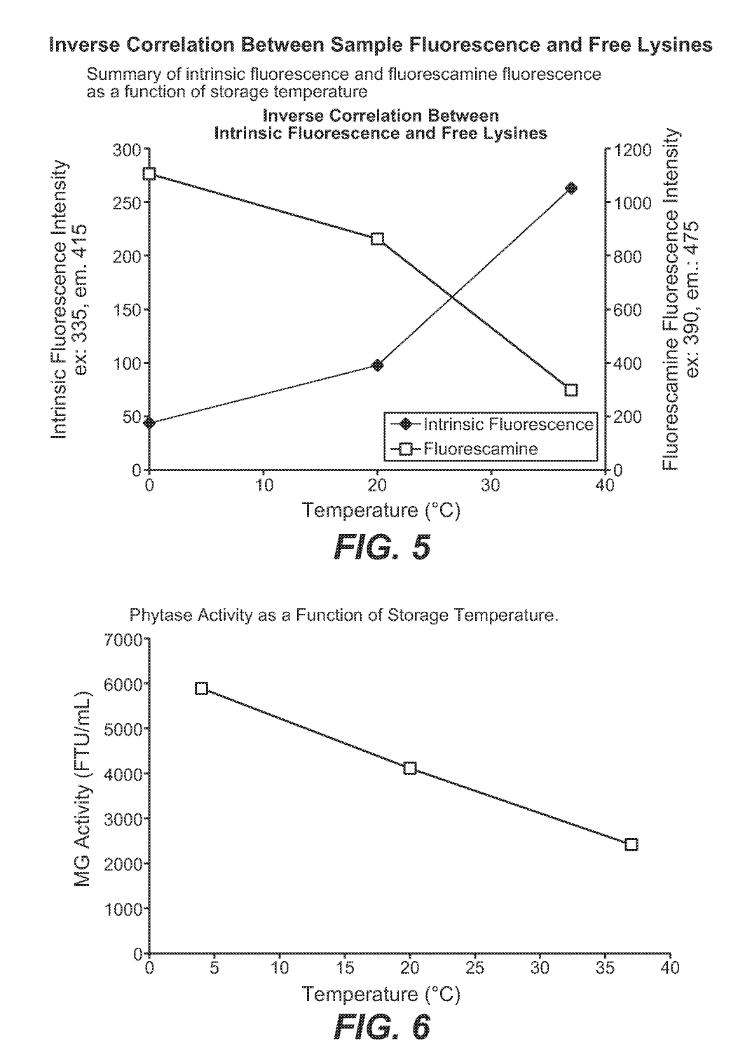

FIG. 5 summarizes the effect of incubation temperature on intrinsic fluorescence intensity and fluorescamine-derived fluorescence intensity. Intrinsic fluorescence intensity at 415 nm corresponds with the formation of Maillard products. Fluorescamine-derived fluorescence intensity at 475 nm is due to the reaction of free amino groups with fluorescamine and therefore, represents a lack of Maillard products. Data were obtained from the experiments noted above and plotted on the same graph to illustrate how Maillard products increase with incubation temperature (intrinsic fluorescence), while free lysines decrease (reflected in the diminished fluorescamine-derived signal). FIG. 6 depicts the measured activities for the same sample incubated at the various temperatures. The activity assay was performed with the malachite green reagent, which couples absorbance at 620 nm with phosphate released from a phytate substrate. The attenuation of activity at increased temperature shown in FIG. 6 tracks with increase in Maillard products displayed in FIG. 5.

### EXAMPLE 5

### Glycation Analysis via Mass Spectrometry

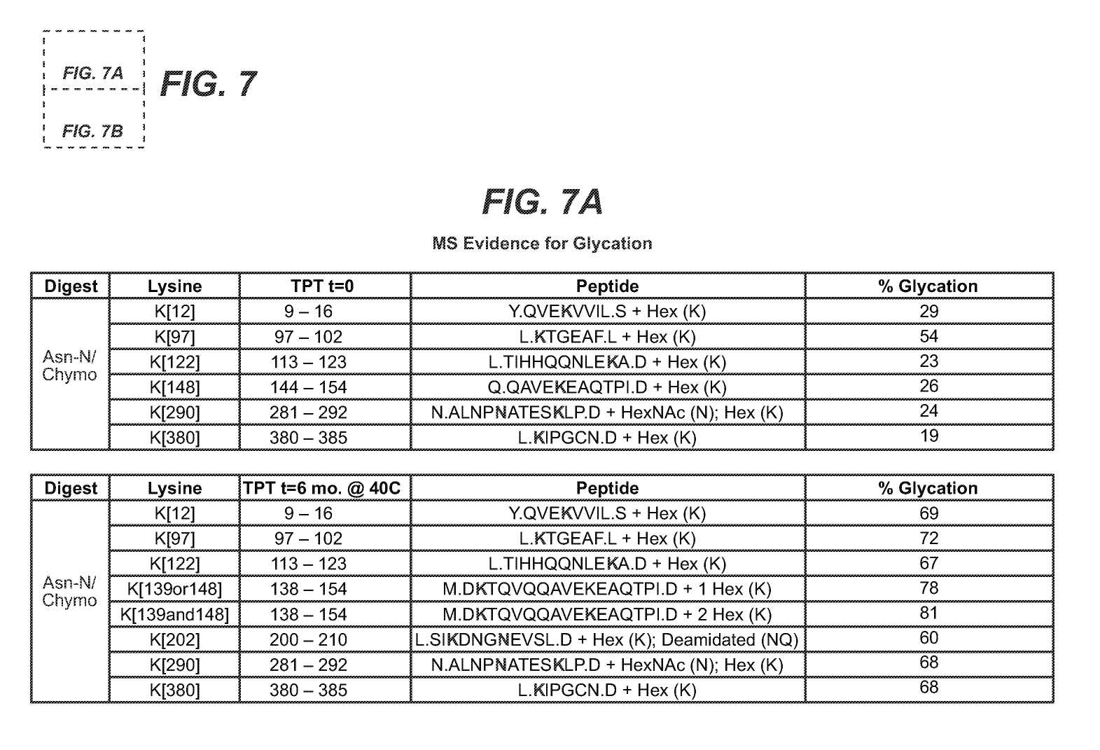

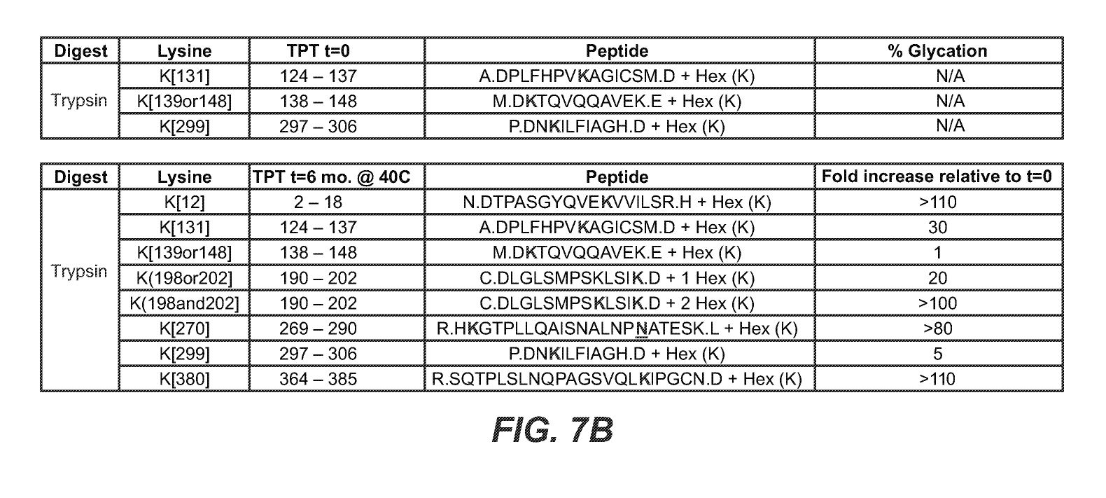

Mass spectrometry evidence for glycation of phytase is shown in FIGS. 7A-B . These data were obtained from an Asp-N endoproteinase/chymotrypsin and trypsin digests with database searches for hexose (i.e. glycation) modification on lysine residues. For the Asp-N/chymotrypsin digest, quantitation was established by obtaining the ratio of glycated vs. non-glycated peptides. It is important to note the quantification numbers are only approximate as glycation of a lysine residue is expected to decrease its ionization efficiency. In the AspN/chymotrypsin digests at time 0, between 19 and 54% of the lysine groups on phytase are glycated. After storage for 6 months at 40 C, the percentage of glycation increases for all lysines increases to between 67 and 81%. Trypsin digests were also performed, but could not be quantified in the same manner due to the fact that this enzyme cleaves at lysine and arginine residues. Accordingly, the fold increase in modification relative to t=0 for the trypsin digest was determined by normalization to an internal standard peptide In the trypsin digest, glycation increase is observed on several lysines by factors ranging up to ˜110-fold.

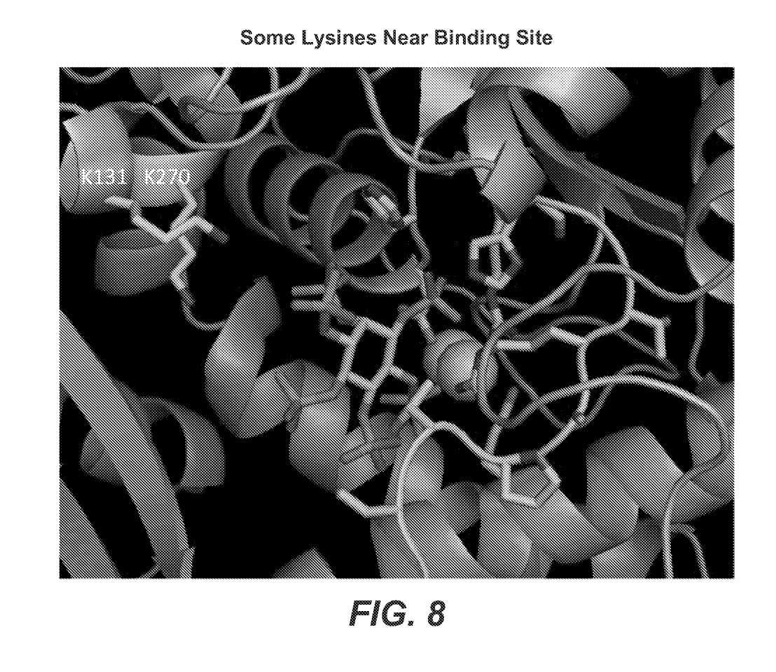

In FIG. 8, the two lysines, K131 and K270, located closest to the active site of phytase are highlighted. Without being bound by theory, it was hypothesized that it is the glycation of one, both, or other lysines near the active site which is responsible for the reduction in activity of phytase towards its natural substrate, phytate while retaining nearly all of its activity against the much smaller synthetic substrate para-nitrophenylphosphate (pNPP).

### EXAMPLE 6

### Induced Glycation in Lyophilized Samples of Purified BP17 ETD

2 mg/ml of BP17 ETD phytase was purified via high ion exchange chromatography and combined with 0 to 0.0625 M of glucose or fructose in solution. These solutions were lyophilized to produce a dry solid and this lyophilized material was incubated at 50° C. and 70% relative humidity for 6 days to induce glycation or the Maillard reaction of the phytase with the added reducing sugar.

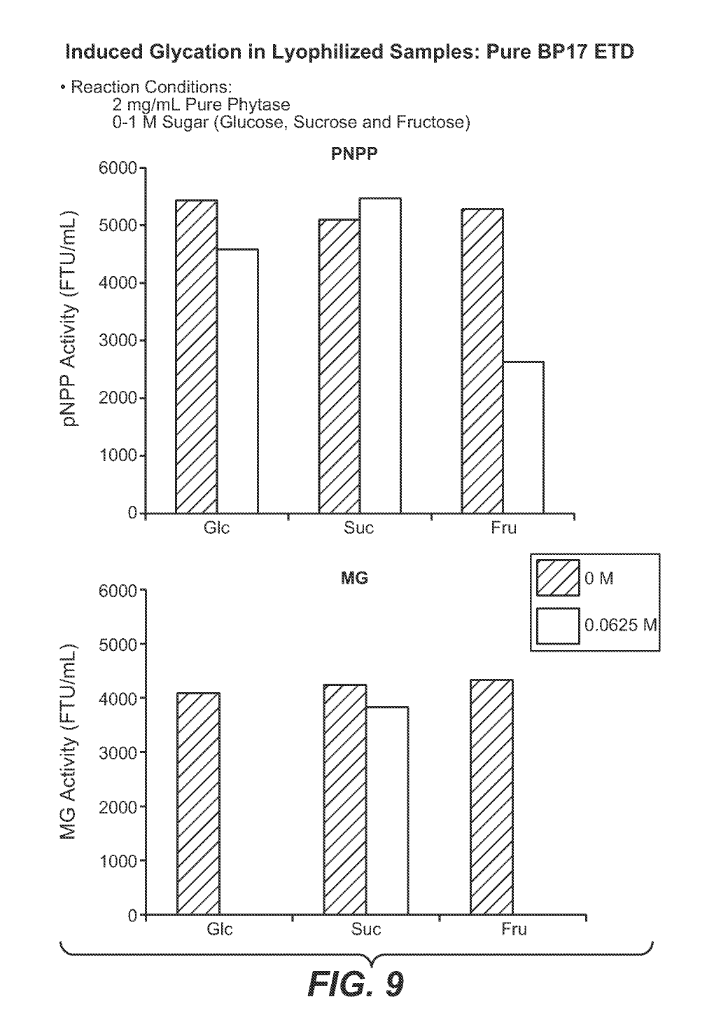

FIG. 9 shows the effect of induced glycation from glucose and fructose after incubating 0.0625 M of the sugar with phytase at 50° C. and 70% relative humidity for 6 days. It can be seen that for glucose the pNPP activity is reduced from 5500 FTU/ml to 4500 FTU/ml while the activity on phytate measured via the MG assay is reduced from 4000 FTU/ml to 0 FTU/ml. Likewise for fructose the activity is reduced from 5200 FTU/ml to 2700 FTU/ml towards pNPP and from 4100 FTU/ml to 0 FTU/ml for fructose. As expected, incubation with the non-reducing sugar, sucrose, did not result in any significant loss of either pNPP or phytase activity. Similar results were obtained for phytase from UFC.

### EXAMPLE 7

### Heat Treatment of BP17 ETD Phytase UFC to Remove Background Activities

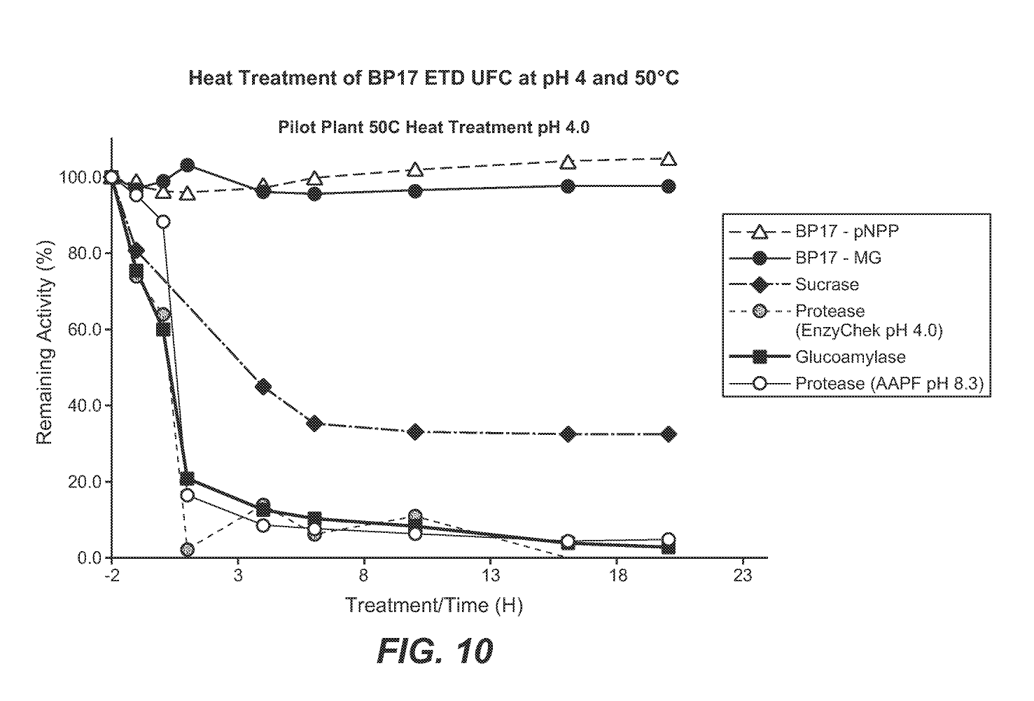

Heat treatment of the BP17 ETD phytase UFC is done to remove background sucrase and glucoamylase activity which can generate glucose during storage from sucrose and starch in the formulation, respectively. FIG. 10 shows an example of a heat treatment process and the corresponding reduction in measured background activities with time of heating. Phytase UFC is heated to 50° C. for 4 to 20 hours at pH 4.0 and this causes glucoamylase activity reduction between 90 and 95% and sucrase activity reduction of about 65%. Protease activity is also reduced by 90-95% but this activity is not believed to be responsible for glucose generation or glycation of the phytase.

### EXAMPLE 8

### Accelerated Storage Stability

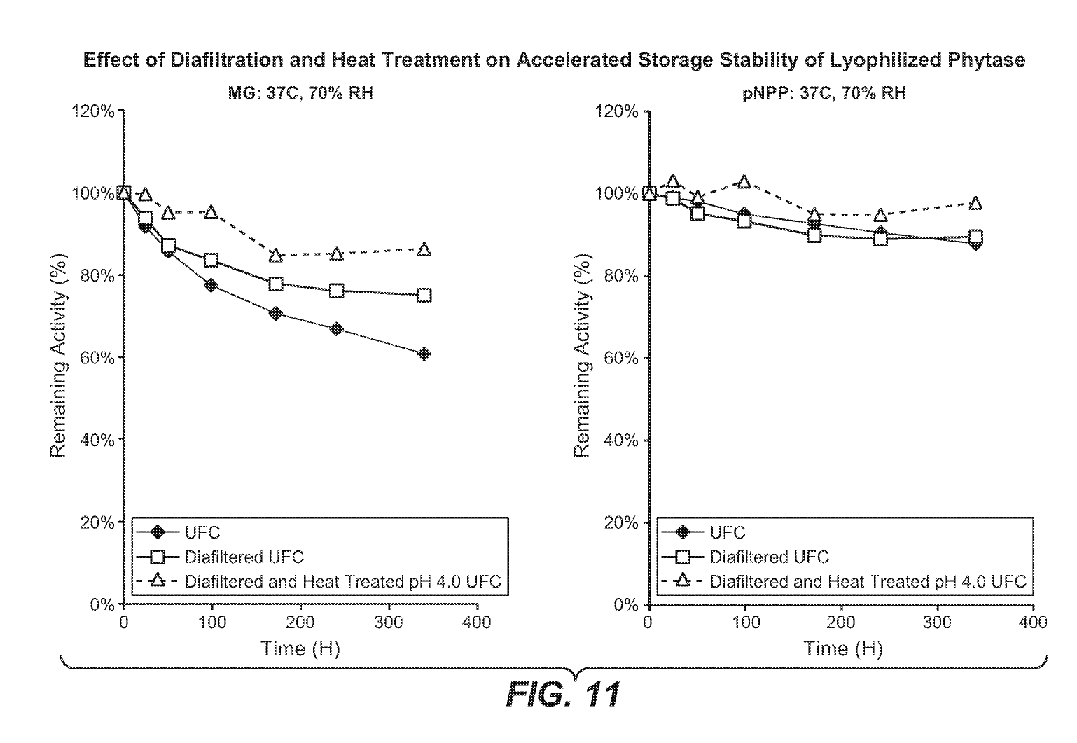

FIG. 11 shows the effect of both diafiltration and heat treatment (50 C for 16 hours) on the retained activity on lyophilized UFC. Diafiltration was carried out with 3 kg of Phytase UFC. The starting UFC had a pH of 4.0 and conductivity of ˜2.7 S/cm. A Millipore unit with 10 kDa Polyethersulphone (PES) Millipore membrane was used for this experiment. The UFC was diafiltered with 3 diavolumes of process water in batch mode at a temperature of 10-15° C. Batch mode here refers to a technique in which 1 diavolume of water is added upfront to the UFC followed by collection of 1 diavolume of UF permeate. This process is repeated 3 times in order to achieve 3 diavolumes of diafiltration. At the end of the diafiltration, if the pH of the UFC has drifted >4.5, 20% acetic acid is used to adjust the pH down to 4.0.

It can be seen that both diafiltration and heat treatment improve the storage stability of the dry phytase towards its natural substrate phytate (MG Assay) while only heat treatment improves the storage stability of the dry phytase towards its synthetic pNPP substrate. This is most likely because diafiltration is primarily removing glucose and other reducing sugars and therefore preventing glycation of the phytase. In contrast, heat treatment is primarily deactivating the background enzymes protease, glucoamylase and sucrase and since no sucrose or starch is present in these UFC's, the heat treatment is primarily preventing proteolysis which affects activity towards both the synthetic and real substrate.

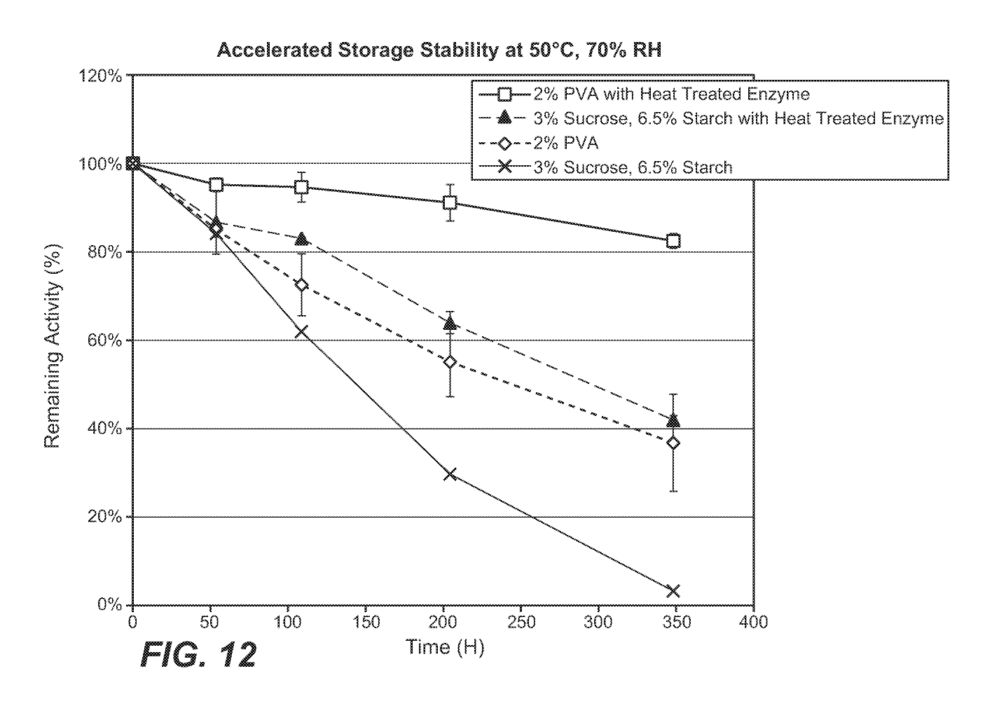

FIG. 12 shows the phytase activity remaining for granules having four different enzyme matrix compositions stored at 50° C. and 70% relative humidity. The granules were compositionally similar to those described in Example 3. For the granule made with 3% sucrose and 6.5% corn starch in the enzyme matrix with untreated enzyme concentrate, it can be seen that only 3% activity is remaining after 350 hours of storage. When 2% PVA is substituted for the sucrose and corn starch in the enzyme matrix, thus getting rid of all internal sources of glucose generation, it can be seen that the activity after 350 hours of storage is now increased to 37%. Similarly, when the sucrose/starch enzyme matrix based granule is made with heat treated enzyme, thus deactivating the enzymes responsible for glucose formation in the enzyme matrix, the retained activity after 350 hours is also increased, this time to 42%. Finally, if the granule is made with both 2% PVA in the enzyme matrix and heat treated enzyme concentrate, the final retained activity after 350 hours of storage is 82%. This is believed due to the deactivation of a protease which occurs during the heat treatment process combined with the removal the glucose forming sucrose and starch substrates.

### EXAMPLE 9

### DXST

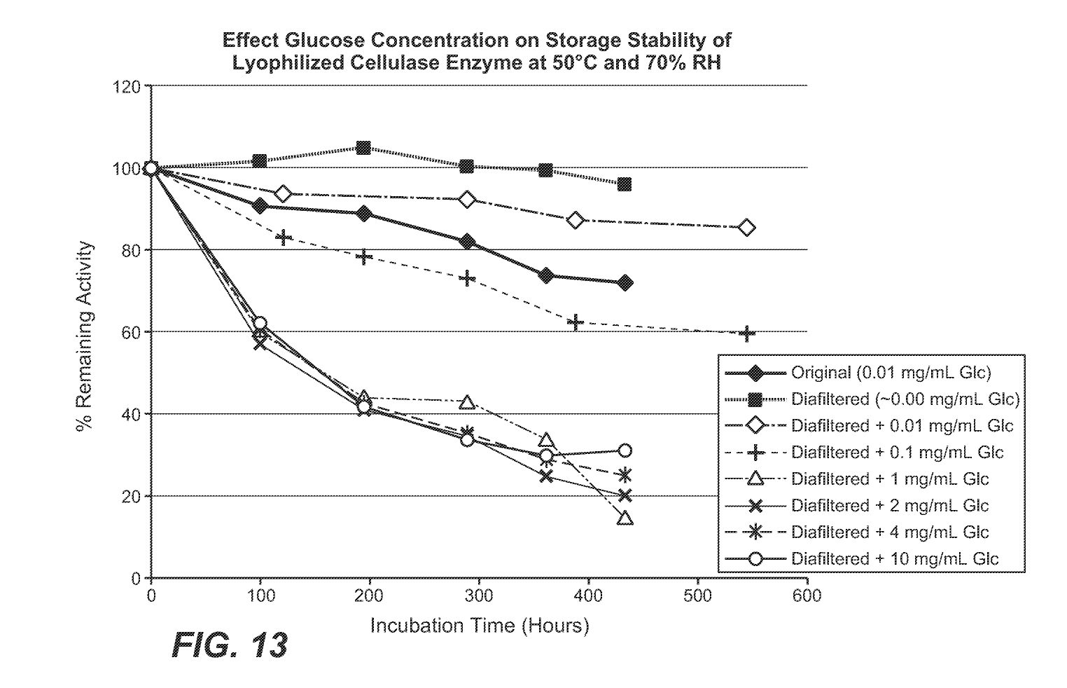

FIG. 13 shows the stability of various lyophilized cellulase enzyme UFC's at 50° C. and 70% RH as a function of glucose concentration. DXST was expressed in Trichoderma resei, its sequence is provided below.

SEQ ID NO: 2
MRSSPVLRTALAAALPLAALAADGKSTRYWDCCKPSCSWPGKASVNQ
PVFACSANFQRISDPNVKSGCDGGSAYACADQTPWAVNDNFSYGFAA
TSISGGNEASWCCGCYELTFTSGPVAGKTMVVQSTSTGGDLGTNHFD
LAMPGGGVGIFDGCSPQFGGLAGDRYGGVSSRSQCDSFPAALKPGCY
WRFDWFKNADNPTFTFRQVQCPSELVARTGCRRNDDGNFPVFTPPSG
GQSSSSSSSSSAKPTSTSTSTTSTKATSTTSTASSQTSSSTGGGCAA
QRWAQCGGIGFSGCTTCVSGTTCNKQNDWYSQCL

The cellulase is an endo-beta-D-1,4-glucanase from Staphylotrichum coccosporum gi|197267671 (short name STCE1). It can be seen that the original UFC containing about 0.01 mg/ml glucose shows about a 30% loss of activity over 431 hours while the lyophilized UFC shows no loss of activity over the same time period. Adding glucose back to the lyphilized UFC causes the celllulase to become unstable again causing as much as 80% loss of activity over 450 hours at glucose concentrations of 1 mg/ml and higher. Interestingly, adding 0.01 mg/ml glucose back to the diafiltered UFC does not cause as much activity loss as the original UFC which contains 0.01 mg/ml glucose. This can be explained by the hypothesis that glucose may not be the only reducing sugar present in the original UFC.

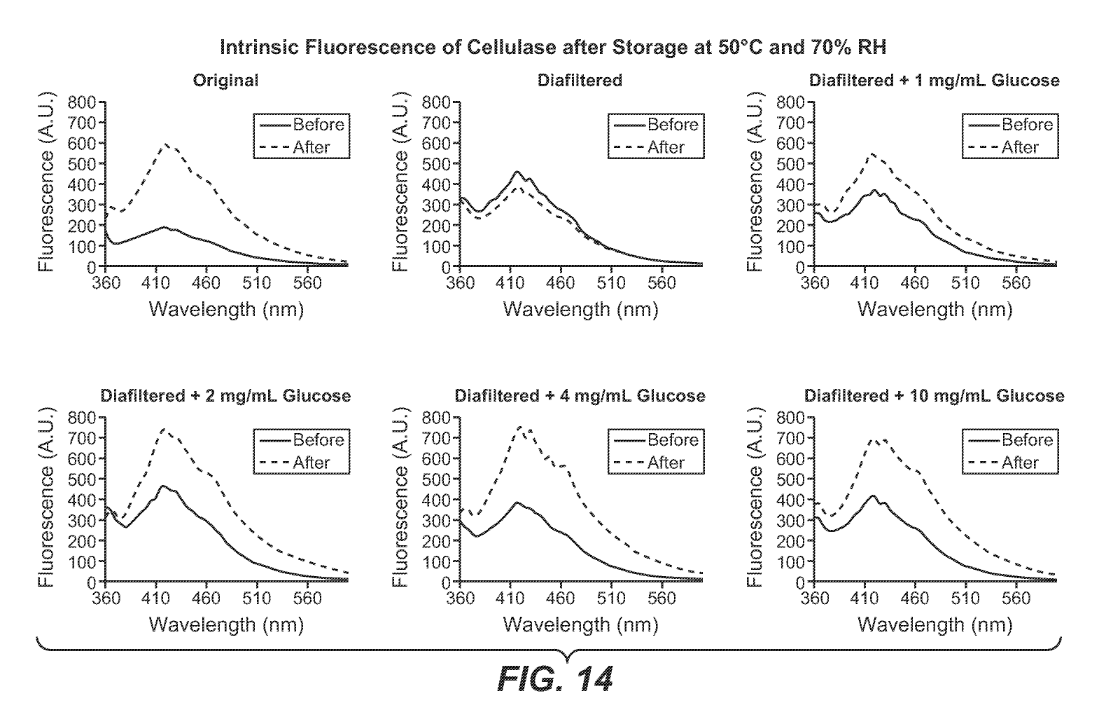

FIG. 14 shows the intrinsic fluorescence of various lyophilized cellulase UFC's (similarly treated samples from FIG. 13) after storage at 50° C. and 70% RH for 431 hours. It can be seen that the original cellulase UFC shows a 300% increase in intrinsic fluorescence while the lyophilized cellulase shows only a 20% increase. Diafilitered cellulase UFC with added glucose shows progressively greater levels of intrinsic fluorescence showing up to a 150% increase in intrinsic fluorescence at the highest level of added glucose.

Without being bound by theory, it is hypothesized that at least one of the lysines near the active site include K25, K34, K42 and K65 may be responsible for this loss.

### EXAMPLE 10

### Protein Engineering Studies

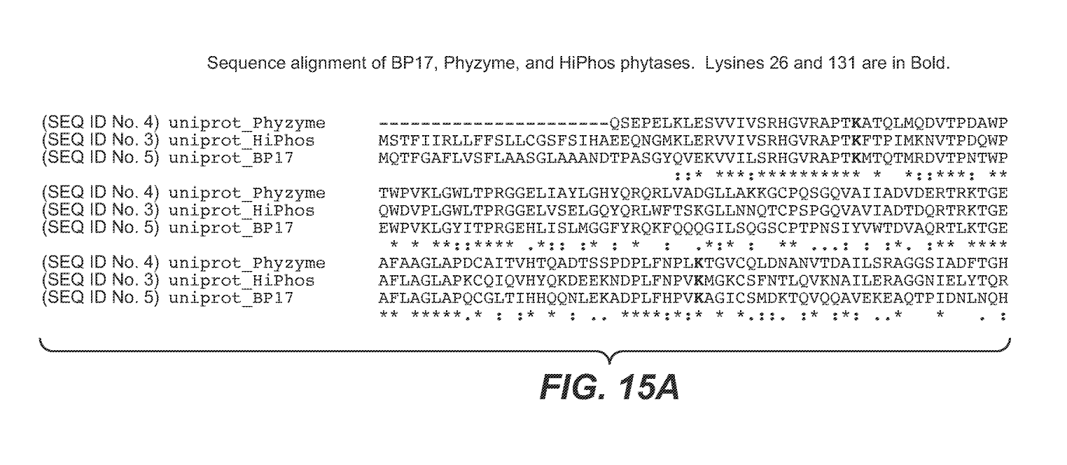

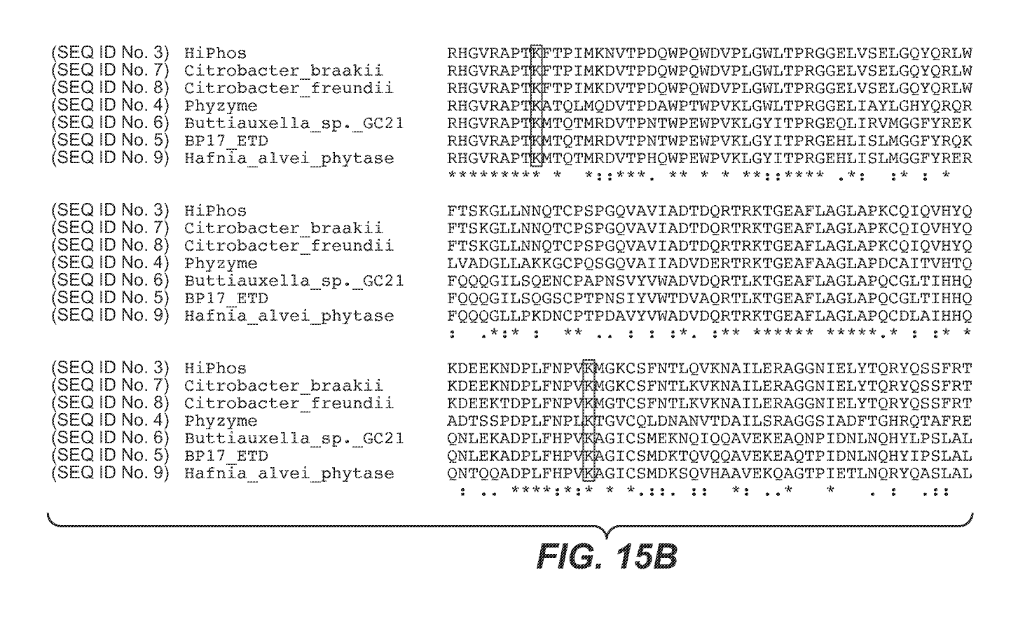

To explore the role of specific lysine amino acids contributing to glycation, and protein stability, protein engineering efforts were undertaken. This study was performed using WT and variants K131R and K26R. These lysine residues were chosen based on (1) conservation at these positions among other phytase sequences (FIG. 15A), (2) proximity to the substrate binding site and (3) results from mass spectral analysis of phytase BP17 from previous studies.

Sequences for the proteins considered in FIG. 15A are below.

>HiPhos
SEQ ID NO: 3
MSTFIIRLLFFSLLCGSFSIHAEEQNGMKLERVVIVSRHGVRAPTKF
TPIMKNVTPDQWPQWDVPLGWLTPRGGELVSELGQYQRLWFTSKGLL
NNQTCPSPGQVAVIADTDQRTRKTGEAFLAGLAPKCQIQVHYQKDEE
KNDPLFNPVKMGKCSFNTLQVKNAILERAGGNIELYTQRYQSSFRTL
ENVLNFSQSETCKTTEKSTKCTLPEALPSELKVTPDNVSLPGAWSLS
STLTEIFLLQEAQGMPQVAWGRITGEKEWRDLLSLHNAQFDLLQRTP
EVARSRATPLLDMIDTALLTNGTTENRYGIKLPVSLLFIAGHDTNLA
NLSGALDLNWSLPGQPDNTPPGGELVFEKWKRTSDNTDWVQVSFVYQ
TLRDMRDIQPLSLEKPAGKVDLKLIACEEKNSQGMCSLKSFSRLIKE
IRVPECAVTE
>Phyzyme
SEQ ID NO: 4
QSEPELKLESVVIVSRHGVRAPTKATQLMQDVTPDAWPTWPVKLGWL
TPRGGELIAYLGHYQRQRLVADGLLAKKGCPQSGQVAIIADVDERTR
KTGEAFAAGLAPDCAITVHTQADTSSPDPLFNPLKTGVCQLDNANVT
DAILSRAGGSIADFTGHRQTAFRELERVLNFPQSNLCLKREKQDESC
SLTQALPSELKVSADNVSLTGAVSLASMLTEIFLLQQAQGMPEPGWG
RITDSHQWNTLLSLHNAQFYLLQRTPEVARSRATPLLDLIMAALTPH
PPQKQAYGVTLPTSVLFIAGHDTNLANLGGALELNWTLPGQPDNTPP
GGELVFERWRRLSDNSQWIQVSLVFQTLQQMRDKTPLSLNTPPGEVK
LTLAGCEERNAQGMCSLAGFTQIVNEARIPACSLRSHHHHHH
>BP17 ETD
SEQ ID NO: 5
MQTFGAFLVSFLAASGLAAANDTPASGYQVEKVVILSRHGVRAPTKM
TQTMRDVTPNTWPEWPVKLGYITPRGEHLISLMGGFYRQKFQQQGIL
SQGSCPTPNSIYVWTDVAQRTLKTGEAFLAGLAPQCGLTIHHQQNLE
KADPLFHPVKAGICSMDKTQVQQAVEKEAQTPIDNLNQHYIPSLALM
NTTLNFSKSPWCQKHSADKSCDLGLSMPSKLSIKDNGNEVSLDGAIG
LSSTLAEIFLLEYAQGMPQAAWGNIHSEQEWALLLKLHNVYFDLMER
TPYIARHKGTPLLQAISNALNPNATESKLPDISPDNKILFIAGHDTN
IANIAGMLNMRWTLPGQPDNTPPGGALVFERLADKSGKQYVSVSMVY
QTLEQLRSQTPLSLNQPAGSVQLKIPGCNDQTAEGYCPLSTFTRVVS
QSVEPGCQLQ

The following materials were used: Buttauxilla sp. phytase variants were produced in Trichoderma reseei host and purified using ion exchange chromatography; BP17/BP111 Konelab Standard (22000 FTU/g; density=1.07); 402.4 mM glucose in water; Zeba 7 kDa Cutoff Desalting Plate; para-nitrophenylphosphate; and, Phytate/Malachite Green Reagents.

To induce the Maillard reaction, glucose stock solutions were prepared at several concentrations: 0, 8, 20 and 40 mM. Reactions were initiated by mixing 50 uL of phytase variant (2 mg/mL) with 50 uL of glucose solution in PCR strips. A PCR strip was prepared for each of four time points. Samples were frozen, lyophilized and placed in an incubator at 40 C and 70% Relative Humidity (RH). Samples were removed at 0, 58, 100 and 237 hours and stored at −20° C. until completion of the study. Afterwards, samples were resuspended with 100 uL of water and processed using a Zeba 7 kDa cutoff desalting plate to remove excess glucose.

Enzyme activities were determined using pNPP and IP6 substrates with BP17 enzyme standards. The pNPP assay relies on formation of the p-nitrophenylate ion, which absorbs at 405 nm. The IP6 assay is dependent on malachite green (MG) staining of the molybdate-phosphate complex, which absorbs at 620 nm.

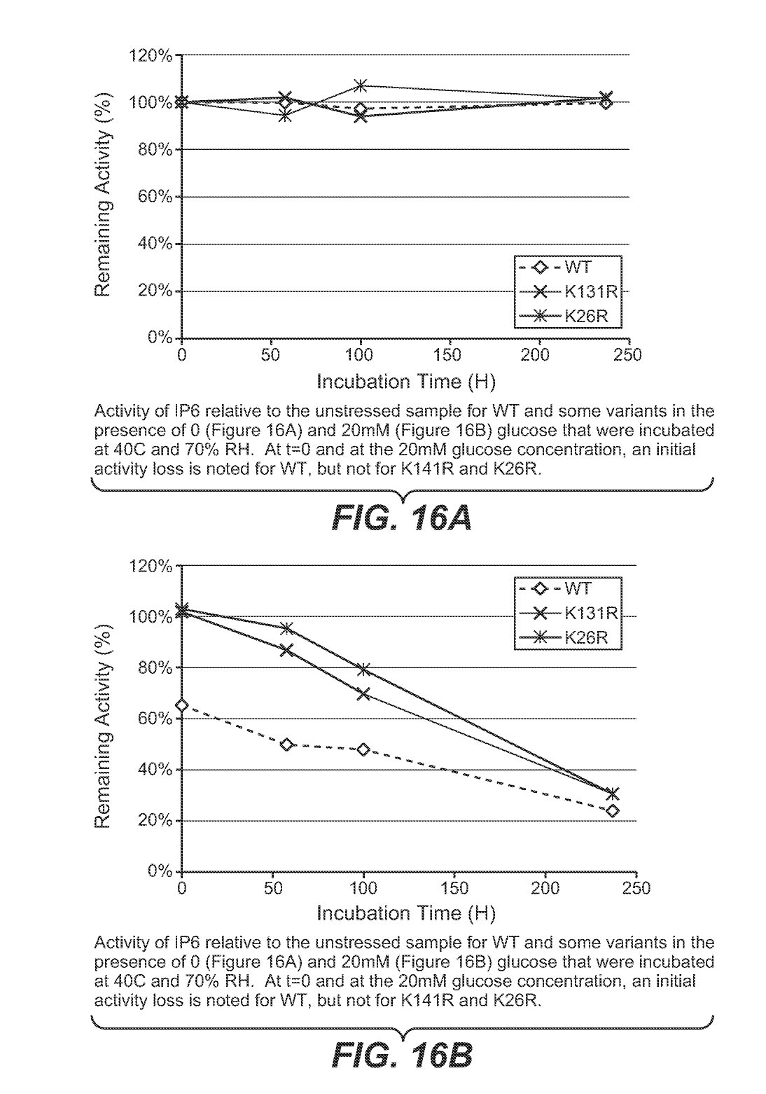

These data indicated that samples not treated with glucose retained full activity for both IP6 (FIG. 16A) and pNPP (not shown) substrates throughout the duration of the stress test at 40 C and 70% RH.

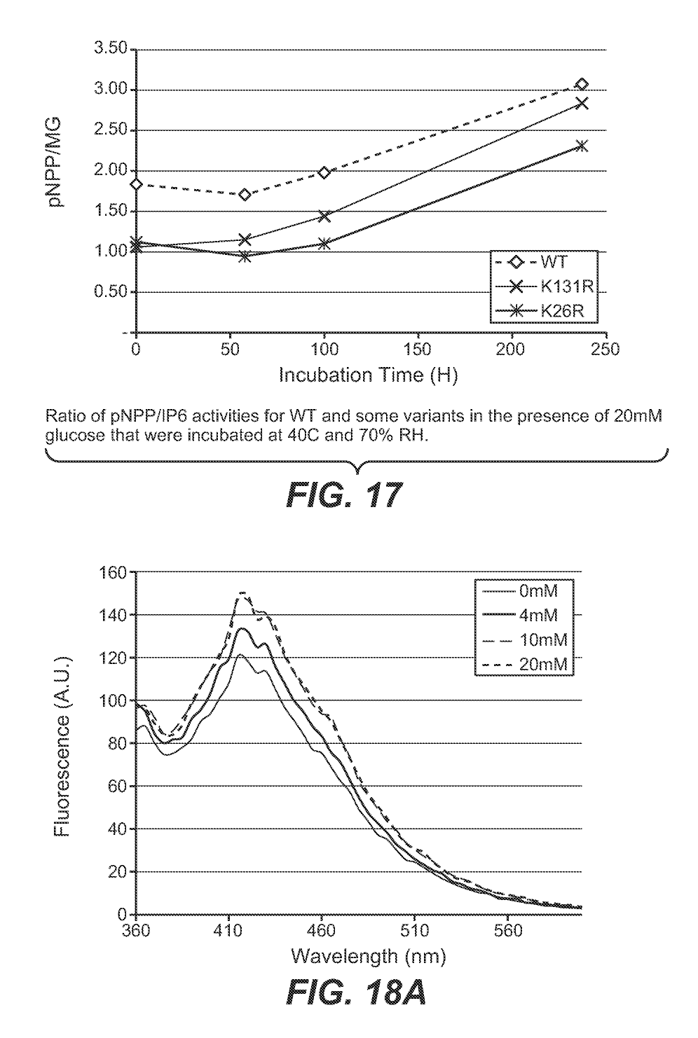

Further, retention of activity toward pNPP and loss of activity toward IP6 is an indication of the extent of lysine glycation due to the Maillard reaction. While the activity on pNPP is retained during glucose stress, activity on IP6 is diminished at initial time points (even at time zero) with an exception of K131 R, and to a lesser extent K26R (FIG. 16B). The increase in the pNPP/MG ratio correlated with the concentration of glucose (FIG. 17).

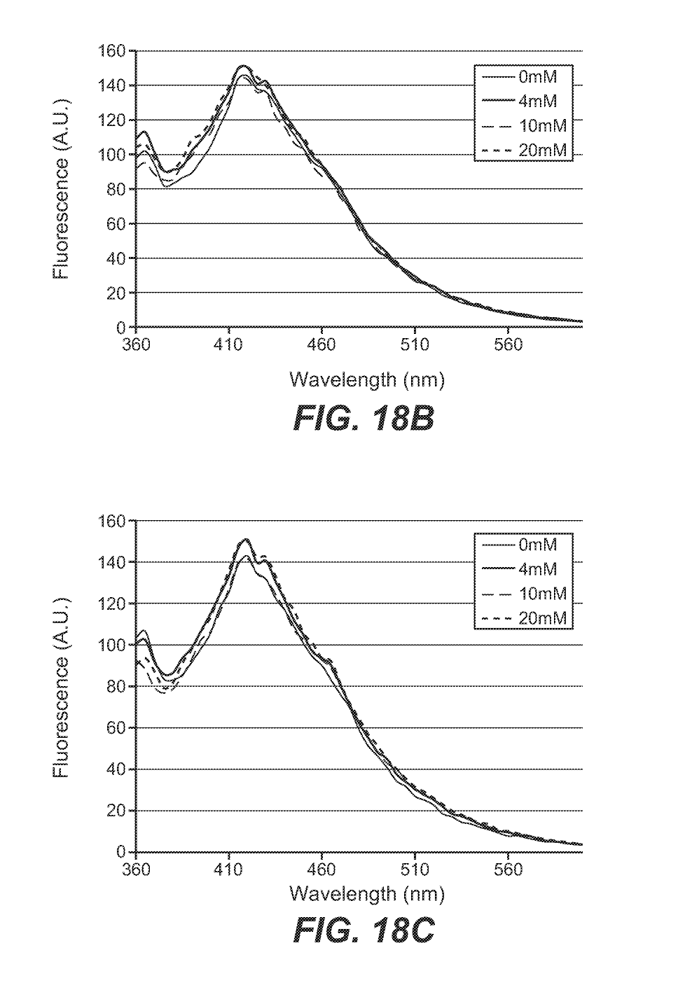

Also, at time 100 h, a glucose-dependent increase of intrinsic fluorescence is noted for WT, but not for K131R and K26R (FIGS. 18A-C). This increase of fluorescence at 415 nm is indicative of AGE formation in WT phytase. Intrinsic fluorescence spectra of WT phytase and its variants is shown in FIG. 18A, K131R (FIG. 18B), and K26R (FIG. 18C), sampled after 100 h at 40° C. and 70% RH. The glucose dependent increase in fluorescence signal at ˜420 nm is a signature of the Maillard reaction (Journal of Dairy Science 94:5375-5380).

These results suggest that K131 and K26 are critical lysines that upon glucose stress alter IP6 substrate specificity. The fact that K131R and K26R variants are resistant to glucose stress while the wild type loses activity toward IP6 suggests that these residues undergo the Maillard reaction.

Additional sequences of interest that will be appreciated to benefit from the present teachings are shown in FIG. 15B, and includes
SEQ ID NO: 6 gi|161898386|gb|ABX80238.1|phytase[Buttiauxella sp.
SEQ ID NO: 7 GC21];>gi|42795098|gb|AAS45884.1|phytase [Citrobacter braakii];
SEQ ID NO: 8 >gi|40748277|gb|AAR89622.1|phytase[Citrobacter freundii]; and,
SEQ ID NO: 9 >gi|364569314|gb|EHM46937.1|histidine acid phosphatase [Hafnia alvei ATCC 51873]

### EXAMPLE 11

### Accelerated Stress Test:

Phytase granules (2 g), prepared according to the process described for Example 1 above, were weighed in individual specimen cups. Seven cups per sample were prepared, each cup representing a time point. Open cups were placed in incubators set to either 40° C. and 70% Relative Humidity (RH), or 50° C. and 70% RH. Cups containing samples were collected from the incubator at 0, 125, 305 and 335 h. Samples were allowed to equilibrate to ambient temperature and humidity for 20 minutes, covered with a lid and stored at −20° C. until all samples were collected.

### Phytase Remaining Activity:

Granules (0.1 g) were extracted with ˜9.9 g assay buffer in 15 mL conical tubes. Samples were allowed to dissolve for 20 min at room temperature with 58 RPM rotation. Samples were centrifuged at 3000 RPM for 3 min, supernatant containing phytase was manually filtered. Phytase activity, for all extracted samples, was assumed to be ˜120 FTU/mL and were diluted with MG assay buffer to 15 FTU/mL for pNPP assay and to 0.010 FTU/mL for IP6/Malachite Green assay.

### Glucose Level in Complete Granules:

Granules were grounded with mortar until a fine powder was obtained. Grinding process was performed in the hood to avoid enzyme exposure. Ground samples (2 g) were weighed in 15 mL conical tubes and 3 mL 2% TFA was added to the dry powder. Sample was mixed and allowed to dissolve for 15 min at room temperature by constant rotation at ˜25 RPM. Sample (500 uL) was neutralized with 500 uL Tris (pH 7.6), mixed, centrifuged for 2 min at 15000 RPM and supernatant was analyzed for glucose using the Konelab glucose oxidase assay.

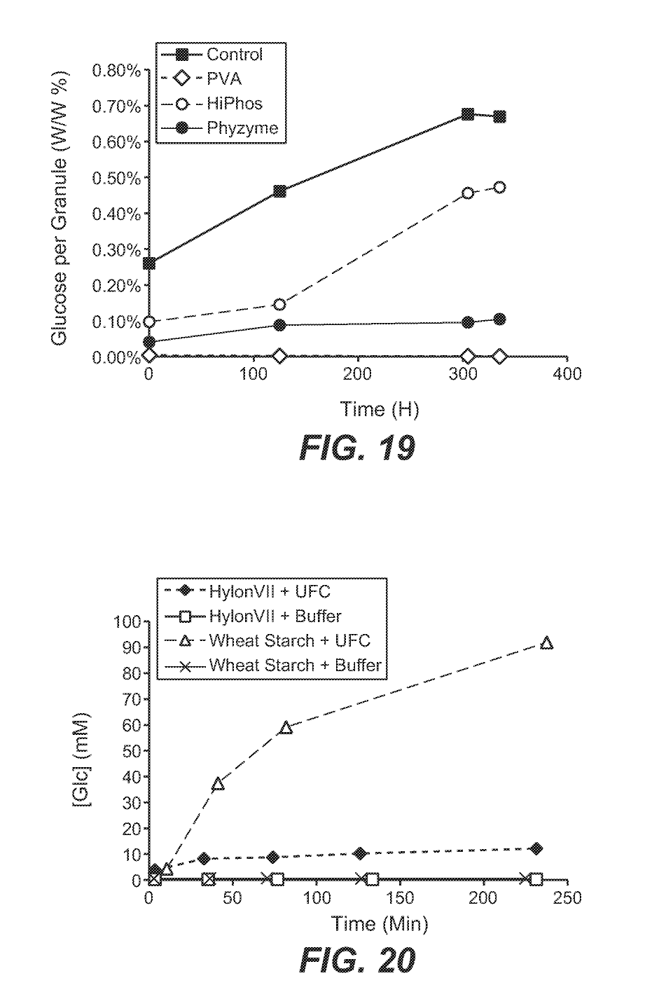

The results are depicted in FIG. 19. Shown here is the concentration of glucose/granule in W/W % produced over time for Phyzyme, Hiphos, and different BP17-containing TPT granules made with diafiltered UFC in the presence of either starch/sucrose (control) or PVA in the enzyme matrix. Values are those for samples collected at 0, 125, 305 and 335 h post accelerated stressed test performed at 40 C and 70% RH.

### EXAMPLE 12

### Resistant Starch as a TPT Formulation Component to Reduce Maillard Reaction

Reducing sugars in solid formulations can be part of the formulation ingredient (e.g., dextrose) or produced inadvertently overtime from other components such as starch and sucrose due to background glucoamylase and invertase. While glucoamylase produces glucose from starch substrate, invertase produces glucose and fructose from the non-reducing sugar sucrose. Glucoamylase and invertase are produced ubiquitously along with the enzyme of interest in the production strain Trichoderma reesei. Thus, using starch and sucrose in solid formulations should be acceptable only if the enzyme of interest can sustain high levels of reducing sugars without losing enzyme activity.

To test the effect of a starch resistant to hydrolysis, glucose production was measured when Nylon VII was employed. Nylon VII is a resistant starch from a corn hybrid that contains 70% amylose and 30% amylopectin. It is predicted that glucose that would normally arise by the background glucoamylase present in diafiltered BP17 ETD UFC, would be reduced by the use of Nylon VII. Measurements were done at 15° C. Total glucose liberated from Nylon VII was compared to that liberated from wheat starch, a more conventional formulation ingredient that would be predicted to undergo hydrolysis by the glucoamylase.

### Materials:

1. Axtra PHY/BP17 ETD UFC from Beloit (C14068; Batch 1681970737) (pH 4.9)

2. 250 mM Sodium Acetate (pH 4.9)

3. Nylon VII Starch

4. Wheat Starch from Hanko

5. Trifluoroacetic acid (TFA)

6. 500 mM Tris, pH 7.6

7. Glucose Oxidase Assay (Konelab)

### Glucose Production:

Axtra PHY/BP17 ETD was diafiltered with three diavolumes. UFC and buffer were pre-chilled on ice prior to initiating reaction. Samples were transferred to a refrigerated incubator set to 15° C. and mixed by an automatic rotator. Wheat starch and Nylon VII (3.7 g) were suspended independently in cold 16.3 g diafiltered UFC or 250 mM sodium acetate (pH 4.9). Starch concentration used is equivalent to that present in TPT formulation. Time points were taken for about 4 h and glucose production was halted by acid quench using TFA. The following time points were collected taking into account the quenching time:

Hylon VII UFC: 3.5, 32.7, 73.87, 126, 231.35 min

Hylon VII Buffer: 3.45, 35.18, 76.65, 133.35, 231.18 min

Wheat Starch UFC: 3.28, 33.9, 74.6, 129.51, 230.26 min

Wheat Starch Buffer: 2.93, 36.27, 70.4, 126.82, 224.67 min

The quench procedure was performed at room temperature as follows. Reaction mixture (1 mL) was spun at 15000 RPM for 2 min. Supernatant (0.49 mL) was collected and added to eppendorf tube containing 10 ul TFA. Solution was vortex to ensure proper mixing and centrifuged for 2 min at 15000 RPM to remove precipitate. Supernatant (400 ul) was added to 500 ul of 500 mM Tris, pH 7.6 in a new tube as a way to neutralize the solution prior glucose measurement. Samples were frozen at −80 C soon after the acid quench step and until glucose analysis.

To measure glucose, samples were thawed for 20-30 min at room temp and glucose concentration was determined following the Konelab glucose oxidase assay procedure. Although all UFC samples were in range of the assay, samples in buffer were below the limit of detection.

We observed that background glucoamylase present in BP17 ETD UFC produces 12 mM glucose from Nylon VII starch after 231 min at 15° C. and pH 4.9. This concentration of glucose produced from Nylon VII is ˜7.7-fold lower than that produced from wheat starch after 237 min.

The results are shown in FIG. 20. Here a comparison is shown between glucose production from Nylon VII corn starch and wheat starch at 15 C. The glucose produced is believed generally to arise by the action of background glucoamylase present in the BP17 ETD UFC. Glucose production was initiated by adding starch to either UFC or buffer (pH 4.9) that had been pre-equilibrated to 15° C. Time points were acid quenched followed by neutralization. Glucose produced in the quenched samples was measured using the glucose oxidase assay.

1. A method of improving enzyme stability comprising;
making an enzyme in a fermentation, wherein the fermentation comprises a fermentation medium comprising problematic sugars and hydrolyzable starch, and wherein the enzyme comprises at least one glycatable amino acid residue and is an enzyme susceptible to problematic glycation;
recovering the enzyme; and,
removing sufficient amounts of the problematic sugars to improve enzyme stability relative to a similarly fermented enzyme lacking the removing.

2. The method of claim 1 wherein the removing comprises diafiltering an ultrafiltrate concentrate to remove the problematic sugars to form an enzyme concentrate.

3. The method of claim 2 wherein the diafiltering comprises at least two, at least three, or at least four, diavolumes, such that the sugars content of the enzyme concentrate is reduced to less than 0.1% w/w.

4. The method of claim 1 wherein the removing comprises heating the enzyme after the fermentation at 50° C. for at least 1, 2, 4, 8, or 12 hours to inactivate any starch-hydrolyzing enzymes present.

5. The method of claim 1 wherein the removing comprises a previous genetic manipulation, wherein at least one active site lysine residue was removed from the polypeptide.

6. The method of claim 5 wherein the active lysine residue is K131 and/or K26, and the enzyme is wild type Buttauxella sp. phytase.

7. The method of claim 5 wherein the active lysine residue is K131 and/or K26, and the enzyme is BP17.

8-12. (canceled)

13. A method of improving an enzyme's stability comprising;
determining the existence of at least one glycatable amino acid residue in an enzyme;
determining whether the enzyme is susceptible to problematic glycation;
making the enzyme so as to ensure glycation is reduced relative to an enzyme made without such ensurances, so as to improve enzyme stability relative thereto.

14. The method of claim 13 wherein the ensurances comprise mutating at least one lysine residue.

15. The method of claim 13 wherein the ensurances comprise treating the enzyme with heat.

16. The method of claim 13 wherein the ensurances comprise formulation the enzyme in a granule comprising resistant starch.

17. The method of claim 13 wherein the ensurances comprise diafiltering the enzyme.

18. (canceled)

19. An enzyme granule comprising an enzyme susceptible to problematic glycation, and, a resistant starch.

20. The enzyme granule according to claim 19 comprising;
a) a sodium sulfate seed,
b) an enzyme layer comprising the enzyme susceptible to problematic glycation, the resistant starch, sodium phytate, and rapeseed oil,
c) a sodium sulfate layer, and,
d) an external layer comprising PVA and talc.

21. The enzyme granule according to claim 19 wherein the enzyme is a phytase.

22. The enzyme granule according to claim 19 wherein the enzyme is wild type Buttiauxella sp.

23. The enzyme granule according to claim 19 wherein the enzyme is BP17, or an amino acid sequence 90%, 95%, 97%, or 99% identical to it.

24. The enzyme granule according to claim 19 wherein the enzyme is a cellulase.

25. The enzyme granule according to claim 19 wherein the enzyme is the cellulase exemplified herein, or an amino acid sequence 90%, 95%, 97%, or 99% identical to it.
